(19) United States
(12) Patent Application Publication
(10) Pub. No.: US 2026/0240755 A1
(43) Pub. Date: Aug. 20, 2026

(54) Use Of Plant Cell Suspension Extracellular Vesicle-Derived Small Rnas As Cosmetic Active Ingredient

(71) Applicant: ACTV BiYOTEKNOLOJi LABORATUVAR SANAYi VE TiCARET ANONiM SiRKETi, Bahcelievler/Istanbul (TR)

(72) Inventors: Aysenur Çalli, Bahcelievler/ Istanbul (TR); Begüm Erkök, Bahcelievler/ Istanbul (TR); Hediye Öztop, Bahcelievler/ Istanbul (TR); Vahap Eldem, Bahcelievler/ Istanbul (TR); Yakup Bakir, Bahcelievler/ Istanbul (TR); Yildiz Bodurlar, Bahcelievler/ Istanbul (TR)

(21) Appl. No.: 19/154,320

(22) PCT Filed: Aug. 14, 2023
(86) PCT No.: PCT/TR2023/095001

§ 371 Date: Aug. 6, 2025

(30) Foreign Application Priority Data
Jul. 24, 2023 (TR) 2023/008707

(51) Int. Cl. A61K 8/9789; A61K 8/14; A61K 8/60; A61Q 7/00; A61Q 19/00; A61Q 19/02; A61Q 19/08

(52) U.S. Cl. CPC A61K 8/9789; A61K 8/14; A61K 8/606; A61Q 7/00; A61Q 19/00; A61Q 19/02; A61Q 19/08; A61K 2800/10; A61K 2800/412; A61K 2800/522; A61K 2800/805

(57) ABSTRACT

The present invention pertains to the method of obtaining a raw material with anti-aging, wound healing, spot-reducing, dark circle-reducing, and anti-hair loss activities for use in cosmetic products. The invention comprises the steps of obtaining H. cordata cell suspensions, adding 0.05-0.5 mM MeJa to the H. cordata cell suspension culture medium on the 12th-18th day of the culture, incubating the culture with MeJA for 60-80 hours, isolating extracellular vesicles (EVs) from the culture medium after incubation, isolating small RNAs from EVs, and encapsulating the EV-derived small RNAs in cationic liposomes. The invention covers the use of individual or combinations of plants, including Houttuynia cordata, Centella asiatica, Cydonia oblonga, Malva sylvestris, Persea americana, Rosa damascena, Helichrysum arenarium, Glycine max, Prunella vulgaris, Symphytum officinale, Malva sylvestris, Scutellaria baicalensis, Pelargonium graveolens, Filipendula ulmaria, Passiflora edulis, Marribium vulgare, Linum tauricum, Lycium barbarum, Papaver rhoeas, Paeonia peregrina, Leontopodium alpinum, Prunus dulcis, Inula helenium, and Psoralea corylifolia, as the plant in the invention.

### TECHNICAL FIELD

The invention relates to a method for producing active ingredient, which enables the utilization of small RNAs derived from plant cell suspension extracellular vesicles as anti-aging, wound healing, anti-dark circle, anti-spot, and anti-hair loss cosmetic ingredients.

### PRIOR ART

The skin forms the largest living organ that protects the body from the external environment, helps regulate temperature and fluid balance, keeps harmful microorganisms and chemicals out, and provides some protection against sunlight. It consists of three layers: the epidermis, dermis, and hypodermis, which are tightly connected to each other. The epidermis is the outermost layer of the skin and includes keratinocytes, melanocytes, Langerhans cells, and Merkel cells. It is a layer that renews the skin, creates color, and protects the body. In the dermis, the second layer of the skin, there are fibroblast cells and extracellular matrix components. In the dermis, the second layer of the skin, there are fibroblast cells and extracellular matrix components. Collagen and elastin in the extracellular matrix constitute the main fibrillar support tissue of the dermis, while proteoglycans, glycoproteins, glycosaminoglycans, and hyaluronic acid form other support structures. In the hypodermis, the lowest layer, thick vessels feed the capillaries, nerves, lymphatic channels, hair follicles, and abundant adipocytes.

Although the skin has a protective role against environmental factors, thanks to its protective layers, it can be insufficient against many internal and external factors, and various cosmetic problems are encountered on the skin. The most common cosmetic problems are wrinkles and skin aging, hyperpigmentation, dark circle formation, acne, hair thinning, and loss, increased skin sensitivity, eczema, and microbiome disruption. With the increase in age, there is a loss of both function and structural stability of the skin due to internal and external factors.

With aging, dermis thickness, glycosaminoglycan amounts, and hyaluronic acid production in the skin, the number of fibroblast cells and collagen synthesis decrease. In addition to these, some changes occur in the dermis layer, such as the stabilization of collagen cross-links, disorganization of collagen bundles, a decrease in skin elasticity due to the degradation of elastin fibers, and a decrease in the water-holding capacity of the skin. As the skin ages, various symptoms are observed, including reduced collagen and elastin expression, inflammation, glycation, compromised skin barrier, diminished blood circulation, and the occurrence of pigmentation or combinations of these factors.

Reactive oxygen species (ROS) are formed by removing an electron from a non-radical atom or molecule or by adding an electron to an atom or molecule. Since they can donate or take electrons from other molecules, ROS act as reducing or oxidizing agents in the organism. Free radicals are produced during the normal metabolism of cells and cause damage to structures such as DNA, protein, and lipid. While free radicals can form during metabolism, they can be stimulated by environmental factors such as UV rays, air pollution, and cigarette smoke. However, with high levels of ROS synthesis, the oxidative balance is disrupted, and cell signaling pathways are adversely affected by this situation. Excessive ROS production causes lipid, protein, carbohydrate peroxidation, DNA damage, and apoptosis. ROS synthesis also increases in conditions such as inflammation, cancer, and neurodegenerative diseases. In addition, the increase in ROS synthesis also affects the formation of many skin-related problems, such as skin aging, the formation of spots on the skin, and hair loss. As age progresses, the antioxidant defense of the metabolism decreases, so ROS species are more effective. With increasing oxidative stress, the synthesis of MMPs (metalloproteinases) that degrade ECM (extracellular matrix) components increases. Activation of MMPs damages collagen and elastin components that provide firmness and flexibility in the skin. As a result, wrinkles, which are signs of skin aging, occur, and the elasticity of the skin decreases.

Senescence, synonymous with aging, refers to the cessation of cell replication due to the termination of somatic cells' division capacity. Fibroblast dysfunction and depletion play a significant role in skin aging. As age progresses, signs of aging appear on the skin, and external factors accelerate the skin aging process. Senescence prevents the replication of cells with damaged DNA, arising in response to harmful stimuli such as telomere shortening, DNA damage, and oncogenic signaling. DNA damage and consequent senescence are triggered by ionizing radiation, chemotherapeutics, genotoxic stress, and oxidative stress. Senescence can occur as a natural process in cells or due to factors causing DNA damage. Senescent cells represent the aging process of cells and can lead to a series of effects in the body;
causing the formation of wrinkles by reducing collagen and elastin synthesis in the skin,
leading to decreased skin elasticity and the formation of deep wrinkles, fine lines, and sagging,
resulting in reduced ability to maintain the skin's moisture balance and leading to irritation and flaking,
causing changes in skin color due to decreased or increased melanin production, resulting in the formation of brown or dark spots, also known as age spots,
causing thinning of the skin and increased sensitivity by depleting essential components such as collagen, elastin, and hyaluronic acid,
reducing skin firmness and elasticity, making the skin appear more saggy and loose,
leading to problems such as facial redness, spider veins, and prominent capillaries due to the weakening and enlargement of blood vessels in the skin. These effects can manifest differently in each individual and can interact with various factors such as genetic factors, lifestyle, and environmental factors such as UV radiation.

The increase in free radicals also stimulates the formation of hyperpigmentation in the skin. Hyperpigmentation is a significant cosmetic problem that leads to the appearance of spots on the skin due to excessive production of a pigment called melanin. Melanin is a pigment that gives color to the skin, and its primary function is to protect the skin from UV radiation. However, excessive exposure to UV radiation, side effects of medications and diseases, acne, and certain skin issues can result in hyperpigmentation and the presence of spots on the skin.

Multiple cellular pathways control melanin synthesis (melanogenesis) in the skin and are hormonally stimulated by alpha-melanocyte-stimulating hormone (α-MSH). Upon activation of this cellular pathway, the synthesis of critical enzymes for melanin production, namely TYR (Tyrosinase), TYRP1 (Tyrosinase-related protein 1), TYRP2 (Tyrosinase-related protein 2), and their regulator MITF (Microphthalmia-associated transcription factor), takes place, resulting in melanin synthesis. The exposure of the skin to UV radiation leads to the formation of free radicals and an increase in oxidative stress. This, in turn, triggers the release of inflammatory cytokines and α-MSH from keratinocytes in the epidermal layer, leading to excessive melanin synthesis in melanocytes and resulting in hyperpigmentation. While free radicals stimulate hyperpigmentation, they can also affect the melanosomes (melanin-containing cellular organelles) in melanocytes, altering melanin distribution. As a result, irregular pigmentation can occur on the skin.

Another significant cosmetic problem is the formation of dark circles around the eyes. Dark circles under the eyes are described as bilateral, round, homogeneous spots (pigment macules) in the infraorbital regions. The eye area forms a unique facial area due to its soft and delicate structure. The under-eye region is thinner and looser compared to other areas because it contains less collagen, elastin, glycosaminoglycans, and superficial lipids. The formation of dark circles can be attributed to various etiological factors, including post-inflammatory hyperpigmentation, melanin accumulation, atopic or allergic dermatitis (eczema), venous congestion, reduced microcirculation, the presence of visible green-blue veins due to the thinness of the under-eye skin, shadows resulting from anatomical deformations such as deep tear trough deformity or fat accumulation, under-eye bags, swelling associated with fat loss, leading to the formation of a blue, purple, or pink color tone, and the presence of a brown band caused by the structural hollow of the under-eye.

One or several combinations of factors, such as the weakening of the connective tissue under the eyes, an increase in oxidative stress, an increase in cytokines and nitric oxide (NO) due to inflammation, leading to the weakening and increased permeability of capillaries, an increase in melanin accumulation, and a decrease in microcirculation can cause the appearance of dark circles. An increase in nitric oxide in the blood vessels under the eyes leads to increased vascularization, and at the same time, increased capillary permeability causes blood to leak into the surrounding tissues, resulting in a bruised appearance.

Hair loss is a widespread cosmetic problem encountered in both women and men. Male pattern baldness, commonly known as androgenetic alopecia, is the most prevalent type. Androgenetic alopecia occurs in individuals genetically predisposed to hormonal changes, primarily in men. It is characterized by thinning hair follicles and hair shedding over time. In this type of hair loss, hair on the forehead usually begins to recede, and the hair on the crown of the head may become sparse or thin. This condition is associated with the increased sensitivity of hair follicles to androgen hormones (male hormones) due to genetic predisposition. Testosterone hormone (T) interacts with the 5 Alpha-Reductase enzyme (5αR) found in hair follicle cells, converting it into Dihydrotestosterone hormone (DHT). The resulting DHT binds to Androgen Receptors (AR) in hair follicle cells and suppresses hair growth. When DHT accumulates in hair follicles, the synthesis of Vascular Endothelial Growth Factor (VEGF) in hair follicles decreases, and the capillaries that nourish the hair follicle shrink and recede, leading to miniaturization of hair follicles and hair loss. Consequently, in addition to hormonal suppression, the hair follicle's nutrition is disrupted, leading to a slowed or complete cessation of hair production. The interaction between epithelial and mesenchymal cells in hair follicles plays a crucial role in follicle morphogenesis and hair development. The dermal papilla, a mesenchymal cell cluster located at the base of the hair follicle, plays a vital role in regulating hair growth and the hair cycle. The β-catenin proteins in dermal papilla cells are regulated by the Wnt signal, which is critical for hair follicle regeneration and the continuity of the anagen phase. Interruption of β-catenin signaling prevents anagen stimulation. Therefore, accessing β-catenin proteins within the cell by applying the candidate active ingredient is important. Activation of the Wnt pathway demonstrates the active compound's ability to stimulate hair growth.

Due to the increasing global aging population and individuals' desire to look younger and more attractive, beauty and personal care have become a significant part of the global economy. As a result, thousands of new products are introduced into the cosmetics market every year. Consumers are becoming increasingly aware of product ingredients, leading to a growing demand for especially natural-based cosmetic products. Consequently, innovative and sustainable technologies are being developed to produce natural active ingredients for cosmetic products. In this regard, in recent years, the use of plant cell culture techniques, also known as plant stem cells, to obtain undifferentiated plant cells and the use of these cells and various extracts has become popular in the cosmetic industry. This technique is used in producing cosmetic active raw materials due to its advantages, such as low plant and water usage requirements, the possibility of production under in vitro culture conditions, and standardizability.

The production of active ingredients using plant cell culture technique involves a series of methods: Firstly, sterilization of plant tissue is performed. After mechanically injuring the sterilized plant particle (explants) with a scalpel, the explants are placed in a nutrient medium containing appropriate growth regulators to induce undifferentiated cells called callus. The principle of the plant cell culture method relies on stimulating meristematic activity in the plant through injury and promoting the formation of undifferentiated cells. The callus cells are then transferred to a liquid nutrient medium and incubated for faster proliferation. Increasing the volume of the liquid nutrient medium makes transferring cells to bioreactor systems and their industrial-scale production possible. Various patent applications exist concerning using cells obtained through the plant cell culture technique as active ingredient in cosmetic products. The current patent applications related to this technique are described below:

According to our research on patent databases, no overlapping application has been found with our invention. However, in the prior art of the technique, various patent applications are related to using cells obtained through the plant cell culture method as raw materials in cosmetic products. The current patent applications associated with this technique are described below:

The patent EP1985280A2 relates to using a product containing undifferentiated plant cells as an active ingredient to protect human stem cells against internal and external stresses.

The application WO2021064173A1 discloses using Leontopodium alpinum plant cells in cosmetic applications to protect the skin's macromolecules, such as proteins, nucleic acids, and lipids, against glycation.

The patent applications WO 2009/151302 and KR 2009-0118877 are related to anti-aging or antioxidant compositions obtained from undifferentiated plant cells derived from Panax ginseng cambium and Taxus plants.

The patent application EP1699423 describes the use of lyophilized undifferentiated plant cells from salt-tolerant plant sources to rejuvenate the skin's appearance.

The patent application WO2011121051 (A2) explains using undifferentiated cells from the argan plant for skin aging, inflammation, and wound healing treatments.

The patent EP1244464 describes using extracts from undifferentiated cells of plants belonging to the Leontopodium genus as ultraviolet filtering agents.

The patent EP 1174120 is related to using lyophilized extracts of undifferentiated cells from the Iradaceae family to stimulate the skin's immune function.

The patent application WO 2005/072697 is associated with using lyophilized undifferentiated plant cells as a cosmetic or pharmaceutical component for protective, regenerating, skin depigmenting, and/or whitening purposes.

None of the abovementioned applications are directed towards using small RNAs found in extracellular vesicles (EVs) released by undifferentiated cells in the nutrient medium as a cosmetic active ingredient, as in our application. Additionally, the use of undifferentiated cells/extracts mentioned in these inventions involves plant cell and intracellular components but does not utilize small RNA molecules that are released into the culture medium and play a significant role in intercellular communication. Unlike other inventions, the patent EP3283092A1 relates explicitly to the cosmetic application of peptides from undifferentiated plant cell suspension in the nutrient medium for skin rejuvenation, focusing only on peptides present in the cell nutrient medium and not encompassing small RNAs.

In recent years, in inventions related to the production of plant-derived active ingredients, along with plant extracts and plant cell culture extracts, plant EVs (Extracellular Vesicles) have also begun to be used. EVs are small lipid structures produced by various cells such as microbial, plant, and human cells. They facilitate cell-to-cell communication by enabling the transfer of encapsulated bioactive molecules such as DNA, RNA, proteins, and lipids (Akers et al., 2013, ‘Biogenesis of extracellular vesicles (EV): exosomes, microvesicles, retrovirus-like vesicles, and apoptotic bodies’, Journal of Neuro-Oncology, 113(1), pp. 1-11; Patil et al., 2019, ‘Exosomes: Biogenesis, Composition, Functions, and Their Role in Pre—metastatic Niche Formation’, Biotechnology and Bioprocess Engineering, 24, pp. 689-70; Raposo et al., 2013, ‘Extracellular vesicles: exosomes, microvesicles, and friends’, The Journal of Cell Biology, 200(4), pp. 373-383). Animal-derived EVs are classified based on their sizes, with those smaller than 100 nm called exosomes, those with sizes ranging from 100 to 1000 nm known as microvesicles, microparticles, or ectosomes, and those larger than 1000 nm referred to as apoptotic vesicles (Bazzan et al., 2021, ‘Critical Review of the Evolution of Extracellular Vesicles' Knowledge: From 1946 to Today’, International Journal of Molecular Sciences, 22(12), p. 6417; Liu and Wang, 2023, ‘A review of the regulatory mechanisms of extracellular vesicles-mediated intercellular communication’, Cell communication and signaling: CCS, 21(1), p. 77).

Inventions related to the use of EVs (Extracellular Vesicles) derived from human cells and stem cells have been found for various purposes, including liver fibrosis (WO2019050071A1), protection against cellular senescence (WO2016104911A1), angiogenesis stimulation (WO2018004143A1), anti-cancer effects (WO2022045813A, CN114686593A, KR1101820264B1), neuro-inflammation treatment (WO2019212371A1), hair growth stimulation (EP2629782A1), anti-aging (CN115444807A, WO2017123022A1), and skin whitening (WO2017123022A1).

There are also studies reporting the therapeutic activities of EVs (Extracellular Vesicles) obtained from plants, in addition to those derived from human cells (Kameli et al., (2021) ‘Plant-Derived Extracellular Vesicles: Current Findings, Challenges, and Future Applications’, Membranes, 11(6); Nemati et al., (2022) ‘Plant-derived extracellular vesicles: a novel nanomedicine approach with advantages and challenges’, Cell communication and signaling: CCS, 20(1), p. 69). Plant-derived extracellular vesicles are nano-sized membrane vesicles released by plant cells and contain lipids, proteins, nucleic acids, and specific pharmacologically active compounds.

There are some patent applications related to the cosmetic use of EVs (Extracellular Vesicles) obtained from plants: EP3354272A1 invention discloses ginseng-derived exosome-like vesicles for skin whitening; WO2017057881A1 invention focuses on anti-hair loss, and WO2017164517A1 invention addresses the use as an anti-aging active ingredient. KR20220019228A document describes antioxidant and anti-aging properties of exosomes derived from broccoli; KR20200002496A document deals with skin whitening effects of exosomes from lemon, cucumber, and potato; KR102413200B1 document outlines cell regeneration, skin barrier strengthening, and moisturizing activities of exosomes from rosemary and lotus plant; and KR102391636B1 document explains the use of Scutellaria baicalensis and Houttuynia cordata exosomes as cosmetic active ingredients for improving skin and hair conditions.

The exosomes mentioned in the inventions are directly obtained from apoptotic fluids of plant tissues and not derived from EVs released by plant cell cultures into the nutrient medium. Additionally, the inventions use only exosomes, whereas, as explained in our application, the isolation of small RNAs from EVs and their use is not addressed.

In addition to patents explaining the isolation of exosomes from apoptotic fluids of plant tissues such as fruits, seeds, leaves, and roots and their use for cosmetic activities, various inventions focus on using exosomes released by plant cell cultures into the nutrient medium:
WO2023022414A1 invention describes using exosomes obtained from lily cell culture for wrinkle reduction, skin elasticity improvement, and skin tone balancing,
WO2022071643A1 invention focuses on using exosomes derived from edelweiss plant cell culture in the nutrient medium for increasing skin elasticity and reducing wrinkles,
KR1102257524B1 invention pertains to applying exosomes obtained from Centella asiatica cell culture in the nutrient medium for enhancing skin elasticity and combating wrinkles,
KR102435006B1 invention addresses using exosomes obtained from Gynostemma pentaphyllum plant cell culture in the nutrient medium for strengthening the skin barrier,
KR1102248258B1 invention describes the anti-aging properties of exosomes derived from green tea cell culture,
KR102419102B1 invention involves the application of exosomes sourced from apple plant cell culture for wrinkle reduction and increased elasticity,
EP3815675A1 invention explains using exosomes obtained from rose cell culture in the nutrient medium to improve skin elasticity and reduce wrinkles,
KR1102248259B1 invention focuses on the anti-aging effects of exosomes derived from soybean cell culture,
KR102445516B1 invention pertains to the anti-aging properties of exosomes sourced from ginseng cell culture,
CN115161256A invention describes using exosomes obtained from Polygonum cuspidatum cell culture for stimulating cell repair,
WO2023022446A1 invention explains the cosmetic application of exosomes derived from Gardenia jasminoides cell culture for enhancing skin elasticity, promoting wound healing, balancing skin tone, brightening, and reducing wrinkles.

In the mentioned inventions, although the active substances are obtained from plant cell culture nutrient medium, only exosomes are used as the active ingredient, and there is no study regarding the isolation and utilization of small RNAs. However, in our invention, H. cordata cell suspension culture-derived extracellular vesicles are used to isolate small RNAs. The obtained small RNAs exhibit anti-aging, wound healing, anti-spot, anti-dark circle, and anti-hair loss effects.

Only a few publications are available in the literature related to the isolation of EVs from plant cell culture:

In a publication where the protein and lipid contents of EVs derived from traditionally cultivated plants and plant cell suspensions were analyzed, it was reported that EVs could be obtained from both materials, and EV isolation from suspension cultures was considered more advantageous in production due to the lower amount of accompanying components (Woith et al., (2021) ‘Plant Extracellular Vesicles and Nanovesicles: Focus on Secondary Metabolites, Proteins and Lipids with Perspectives on Their Potential and Sources,’ International Journal of Molecular Sciences, 22(7), p. 3719). However, this publication does not include a study on the isolation of small RNAs from the obtained EVs and their activities (Kocholata et al., (2022) ‘Comparison of two isolation methods of tobacco-derived extracellular vesicles, their characterization and uptake by plant and rat cells’, Scientific Reports, 12(1), p. 19896).

Plant-derived EVs are cellular structures surrounded by a double lipid membrane that plays a role in transferring intercellular materials such as small RNAs, mRNA, long non-coding RNAs, proteins, and lipids. It has been mentioned in the literature that plant EVs are particularly enriched with post-transcriptional regulatory molecules, especially small RNAs (Baldrich et al., (2019) ‘Plant Extracellular Vesicles Contain Diverse Small RNA Species and Are Enriched in 10- to 17-Nucleotide “Tiny” RNAs’, The Plant Cell, 31(2), pp. 315-324; Cai et al., (2021) ‘Message in a Bubble: Shuttling Small RNAs and Proteins Between Cells and Interacting Organisms Using Extracellular Vesicles,’ Annual review of plant biology, 72, pp. 497-524; Zand Karimi et al., (2022) ‘Arabidopsis apoplastic fluid contains sRNA- and circular RNA-protein complexes that are located outside extracellular vesicles,’ The Plant Cell, 34(5), pp. 1863-1881). Small RNAs are crucial signaling molecules, about 21-24 nucleotides long, involved in various processes such as plant development, reproduction, and defense. Due to differences in their biogenesis and action types, small RNAs are divided into two categories: microRNAs (miRNAs) and small interfering RNAs (siRNAs) (Baldrich et al., (2019) ‘Plant Extracellular Vesicles Contain Diverse Small RNA Species and Are Enriched in 10- to 17-Nucleotide “Tiny” RNAs’, The Plant Cell, 31(2), pp. 315-324).

It has been reported that small RNAs carried by plant-derived EVs can suppress the expression of different genes through trans mod (cross-species RNAi) (Cai et al., (2019) ‘Small RNAs and extracellular vesicles: New mechanisms of cross-species communication and innovative tools for disease control,’ PLoS Pathogens, 15(12), p. e1008090; Chen and Rechavi, (2022) ‘Plant and animal small RNA communications between cells and organisms,’ Nature Reviews. Molecular Cell Biology, 23(3), pp. 185-203). Furthermore, it has been determined that certain small RNA types, particularly microRNAs, from plant sources can enter human cells and suppress the expression levels of certain genes (Dad et al., (2021) ‘Plant Exosome-like Nanovesicles: Emerging Therapeutics and Drug Delivery Nanoplatforms’, Molecular Therapy: The Journal of the American Society of Gene Therapy, 29(1), pp. 13-31; Perut et al., (2021) ‘Strawberry-Derived Exosome-Like Nanoparticles Prevent Oxidative Stress in Human Mesenchymal Stromal Cells’, Biomolecules, 11(1), p. 87; Saiyed et al., (2022) ‘Recent trends in miRNA therapeutics and the application of plant miRNA for prevention and treatment of human diseases’, Future Journal of Pharmaceutical Sciences, 8(1), p. 24). In this context, as stated in the application form, small RNAs have been isolated from EVs synthesized by Houttuynia cordata cell suspensions. The isolated small RNAs have been encapsulated in cationic liposomes to be readily used as raw material in cosmetic products.

The inventions related to the isolation and use of small RNAs are described below: US2021155921A1 application concerns the method of obtaining a small RNA-rich Anethum graveolens aqueous extract and is not related to the cosmetic use of small RNAs.

CN101601635A invention is related to using small RNA/DNA in skin whitening lotions and skin care creams. The small DNA/RNAs used in the invention are obtained through oligonucleotide synthesis and are not derived from plants or undifferentiated plant cells.

R102268324B1 application is related to a topical composition containing low molecular weight RNAs enriched with Lilium tigrinum extract. The invention is associated with a cosmetic application that enhances cell viability, protects against PM and DNA damage, and reduces cellular aging.

CA3005605A1 application pertains to a Tiger Lily extract containing small RNAs and its use in anti-aging cosmetics.

CN111956519A application is concerned with using synthetic small nucleic acids that can quickly and safely eliminate freckles as a skincare product.

The inventions mentioned concerning the use of small RNAs are directed toward the utilization of small RNAs obtained from plants, but both of them are not related to the isolation and use of small RNAs from undifferentiated plant cells or small RNAs present in the EVs of cells in the culture medium.

In their review, Zhang et al. (2013, ‘Use of small RNA as antiaging cosmeceuticals,’ Journal of cosmetic science, 64(6)) discussed studies related to the antiaging activities of synthetic small RNAs targeting specific single genes. However, the review does not mention any study on isolating or using small RNAs from plants or plant cells. Oger et al. (2019, ‘Plant Small RNAs: A New Technology for Skin Care,’ Journal of cosmetic science, 70, pp. 115-126) reported on the antiaging activities of an extract enriched with small RNAs obtained through patented (US2021155921A1) extraction processes called Plant Small RNA (PSR) from baobab tree seeds. The study does not provide the details of the small RNA enrichment process. The small RNAs mentioned in the study were not obtained from plant cell cultures or EVs.

It is known that plant small RNA regulator molecules influence gene expression and various cellular pathways in human cells. As described above, some inventions have addressed using small RNAs found in plants for various applications. However, a fundamental limitation of these inventions is the oversight of the developmental stage of the plant from which the small RNAs are isolated. The profile and gene expression dynamics of small RNAs in plants vary significantly depending on the developmental stage and tissue type (such as root, stem, leaf, tuber, bulb, stolon, etc.). Therefore, for cosmetic use, obtaining small RNAs from the plant at the appropriate developmental stage and in sufficient quantities is crucial for practicality and scalability. In the traditional cultivation of plants, the developmental stage can vary due to changing environmental conditions, making it challenging to standardize the amount and profile of small RNAs.

Inventions related to the isolation and use of small RNAs from plants or plant extracts involve direct use of the plant, leading to challenges such as seasonal availability, the need for large quantities of plants and water, and risks of contamination and pathogens. Moreover, as plant tissues contain EVs in the apoplastic fluid, additional steps are required to isolate the EVs, including the fragmentation and removal of plant tissues. Consequently, the purity and yield of EVs obtained from plants are relatively low.

In the inventions describing the extraction of plant extracts with increased small RNA content, no method is disclosed for protecting small RNAs from degradation. When small RNA molecules are purified, they can be affected and degraded by various external factors such as temperature, humidity, and RNAase. When used without being encapsulated in a protective layer, the structures of small RNAs can be compromised, leading to reduced efficiency. Therefore, another disadvantage of these inventions is the inability to preserve the small RNAs obtained after purification.

In conclusion, due to the drawbacks and challenges encountered in prior art, there is a need for further developments in the relevant technical field.

### STATEMENT OF OBJECTIVE OF THE INVENTION

The general purpose of the invention is to address the requirements mentioned above, eliminate all disadvantages present in the prior art, and bring additional advantages by using plant cell suspension extracellular vesicle-derived small RNAs with anti-aging, wound-healing, anti-spot, anti-dark-circle, and anti-hair loss activities as cosmetic raw materials.

The invention aims to solve all the drawbacks existing in the prior art.

The main objective of the invention is to leverage the beneficial effects of plant cell suspension EV small RNAs on the skin and scalp based on data showing that small RNAs obtained from plants suppress gene expression and direct various cellular pathways in animal cells.

Another essential purpose of the invention is to significantly enhance the use of small RNAs in the cosmetic field. Firstly, dedifferentiated plant cells were used instead of traditional plant extracts. The small RNA profile and quantity of dedifferentiated plant cells differ from differentiated plant tissues (such as leaves). Moreover, with our invention, all environmental conditions of plant cell cultures can be standardized, ensuring that small RNAs are consistently obtained at the same developmental stage and in the same quantity.

Another significant objective of the invention is to eliminate the issues associated with traditional cultivation-based methods, such as the requirement for large quantities of plants and water, seasonal availability, and risks of contamination and pathogens. In the current invention, extracellular vesicles can be obtained from cell cultures created using only a few plant parts. Additionally, as plant cells release EVs into the culture medium, there is no need for an isolation step from among plant tissues (such as apoptotic fluid). This simplifies the isolation process and increases the yield of EVs.

Another essential objective of the invention is to preserve the stability of the obtained small RNAs. For this purpose, small RNAs obtained from EVs have been encapsulated using the cationic liposome method. This ensures that the small RNAs remain intact within the formulation without degradation. Liposomes are essentially vesicles composed of one or more layers of phospholipids that encapsulate an aqueous environment (Malheiros et al., (2010) ‘Food applications of liposome-encapsulated antimicrobial peptides,’ Trends in Food Science & Technology, 21(6), pp. 284-292). Due to the hydrophilic head and hydrophobic tail of phospholipids, liposomes possess both hydrophilic and hydrophobic characteristics (Wagner and Vorauer-Uhl, (2011) ‘Liposome technology for industrial purposes’, Journal of Drug Delivery, 2011, p. 591325). The advantage of liposomes over other encapsulation technologies (spray drying, extrusion, etc.) lies in the stability they provide to water-soluble materials in high-water activity applications. Additionally, it is known that liposomes enhance the skin permeability of active ingredients. Studies have reported that small RNAs, such as miRNA (microRNA) and siRNA (small interfering), encapsulated with cationic liposomes, significantly increase their transmission to cells and intracellular expression. In our invention, small RNAs obtained are encapsulated with cationic liposomes, thereby protecting them from degradation and facilitating their transmission into cells.

Another important distinction of our invention from other prior art is the elicitor application, which ensures a high yield of small RNA synthesis in H. cordata suspension cells compared to the control group. Additionally, there needs to be an application in the existing patent applications aiming to increase small RNA synthesis.

### DESCRIPTION OF THE FIGURES

FIG. 1; The growth phase graph of H. cordata cell suspension.

FIG. 2; The evaluation of the effect of elicitor applications on H. cordata cell suspension biomass. SA: Salicylic acid, MeJA: Methyl jasmonate, Cold (4° C.): Cells incubated at 4° C., Hot (40° C.): Cells incubated at 40° C.
FIG. 2.1; Effect of SA, MeJA, and Nutrient Deprivation,
FIG. 2.2; Effect of Cold and Hot applications.

FIG. 3; EVs derived from H. cordata cell suspension culture medium.
Şekil-3.1; FIG. 3.1; NTA (Nanoparticle tracking analysis) measurement result, S-TEM image,
FIG. 3.2; B) Magnification: 60,0000×,
FIG. 3.3; C) Magnification: 120,000×.

FIG. 4; Evaluation of the effect of elicitor applications on the amount of small RNA obtained from H. cordata cell suspension EVs. SA: Salicylic acid, MeJA: Methyl jasmonate, Cold (4° C.): Cells incubated at 4° C., Hot (40° C.): Cells incubated at 40° C.
FIG. 4.1; Effect of SA, MeJA, and Nutrient Deprivation,
FIG. 4.2; Effect of Cold and Hot applications.

FIG. 5; Readings obtained from Small RNA Sequencing reaction,
FIG. 5.1; Phred score,
FIG. 5.2; Single-end read length,
FIG. 5.3; Adapter content,
FIG. 5.4; Sequencing depth,
FIG. 5.5; Sequence contamination graphs.
H_cordata celi: Small RNA obtained from H. cordata cell suspension culture medium. H_cordata_leaf: Small RNA obtained from mature H. cordata leaf.

FIG. 6; Effect of H. cordata cell suspension extract on cell viability. H.E: Concentrations (μg/mL) of H. cordata cell suspension extract. *: indicates a significant difference compared to the control, p<0.0001.

FIG. 7; Effect of liposome-encapsulated small RNAs on cell viability. L.E small RNA: Concentrations of liposome-encapsulated small RNAs derived from H. cordata cell suspension culture medium EVs. (D1=1, D2=5, D3=25, D4=50, and D5=100 μg/mL) *: indicates a significant difference compared to the control, p<0.0001.

FIG. 8; Effect of the composition of H. cordata cell suspension extract and liposome-encapsulated small RNAs on cell viability. H.E and L.E small RNA: Concentrations of H. cordata cell suspension extract and liposome-encapsulated small RNA composition (D1=50 and D2=100 μg/mL). *: indicates a significant difference compared to the control, p<0.0001.

FIG. 9; The relationship between H.E, L.E small RNA, and combined application of H.E and L.E small RNA with anti-aging activity.
FIG. 9.1; COL1A1,
FIG. 9.2; COL3A1,
FIG. 9.3;TGFβ1 gene expression, D1=50 and D2=100 μg/mL, *: indicates a significant difference compared to the control, p<0.0001.

FIG. 10; The relationship between H.E, L.E small RNA, and combined application of H.E and L.E small RNA with wound healing activity.
FIG. 10.1; VEGF,
FIG. 10.2; NFKB,
FIG. 10.3; TNFα gene expression, D1=50 and D2=100 μg/mL. *: indicates a significant difference compared to the control, p<0.0001.

FIG. 11; The relationship between H.E, L.E small RNA, and combined application of H.E and L.E small RNA with hair loss prevention activity.
FIG. 11.1; 5α Reductase,
FIG. 11.2; β-catenin gene expression, D1=50 and D2=100 μg/mL, indicates a significant difference compared to the control, p<0.0001.

FIG. 12; The relationship between H.E, L.E small RNA, and combined application of H.E and L.E small RNA with anti-aging activity.
FIG. 12.1; COL1A1,
FIG. 12.2; COL3A1,
FIG. 12.3; TGFβ1 protein expression, D1=50 and D2=100 μg/mL. *: indicates a significant difference compared to the control, p<0.0001.

FIG. 13; The relationship between H.E, L.E small RNA, and combined application of H.E and L.E small RNA with wound healing activity.
FIG. 13.1; Effect on VEGF expression,
FIG. 13.2; Effect on NFKB expression,
FIG. 13.3; Effect on TNFα protein expression, D1=50 and D2=100 μg/mL. *: indicates a significant difference compared to the control, p<0.0001.

FIG. 14; Effect of the combined application of H.E, L.E small RNA, and H.E and 30 L.E small RNA on melanin content, D1=50 and D2=100 μg/mL. KA: Kojic acid doses (μM). *: indicates a significant difference compared to the control, p<0.0001.

FIG. 15; Effect of the combined application of H.E, L.E small RNA, and H.E and L.E small RNA on tyrosinase activity. D1=50 and D2=100 μg/mL. KA: Kojic acid doses (μM). *: indicates a significant difference compared to the control, p<0.0001.

FIG. 16; Effect of the combined application of H.E, L.E small RNA, and H.E and L.E small RNA on cell senescence.

FIG. 17; Protective effect of the combined application of H.E, L.E small RNA, and H.E and L.E small RNA against DNA damage.

FIG. 18; Effect of the combined application of H.E, L.E small RNA, and H.E and L.E small RNA on intracellular reactive oxygen species (ROS) formation.

FIG. 19; Effect of the combined application of H.E, L.E small RNA, and H.E and L.E small RNA on inflammation-induced nitrite formation. D1=50 and D2=100 μg/mL. LPS: Lipopolysaccharide. *: indicates a significant difference compared to the control, p<0.0001.

### A DETAILED DESCRIPTION OF THE INVENTION

The invention involves obtaining a raw material with anti-aging, wound-healing, anti-spot, anti-dark circle, and anti-hair loss for skin and hair care products. The invention is obtained through the steps of obtaining Houttuynia cordata cell suspensions, adding 0.05-0.5 mM MeJA to the H. cordata cell suspension culture medium on the 12th-18th day of culture, incubating the culture with MeJA for 60-80 hours, isolating EVs from the culture medium after incubation, isolating EV-derived small RNAs, and encapsulating EV-derived small RNAs in cationic liposomes. In a preferred embodiment of the invention, H. cordata cell suspension is used. Alternatively, cell suspensions obtained from individual or combinations of Centella asiatica, Cydonia oblonga, Malva sylvestris, Persea americana, Rosa damascena, Helichrysum arenarium, Glycine max, Prunella vulgaris, Symphytum officinale, Malva sylvestris, Scutellaria baicalensis, Pelargonium graveolens, Filipendula ulmaria, Passiflora edulis, Marribium vulgare, Linum tauricum, Lycium barbarum, Papaver rhoeas, Paeonia peregrina, Leontopodium alpinum, Prunus dulcis, Inula helenium, and Psoralea corylifolia plant species can also be used.

The general aim of the invention is to provide a raw material that fulfills the mentioned requirements, eliminates all disadvantages, and brings additional advantages, particularly for use in the cosmetic sector and skin and hair care products, with anti-aging, wound-healing, anti-spot, anti-dark circle, and anti-hair loss activities, prepared using plant cell suspension extracellular vesicle-derived small RNAs.

The main objective of the invention is to benefit from the cosmetic activities of EV-derived small RNAs from cell suspension culture medium, based on data suggesting that small RNAs obtained from plants suppress gene expression levels and direct various cellular pathways in animal cells.

The invention is a method for producing a raw material with anti-aging, wound-healing, anti-spot, anti-dark circle, and anti-hair loss activities, to be used in the cosmetic sector and skin and hair care products. Its characteristic features are as follows:
obtaining H. cordata cell suspensions,
adding 0.05-0.5 mM MeJA to the H. cordata cell suspension culture medium on the 12th-18th day of culture,
incubating the culture for 60-80 hours,
isolating EVs from the culture medium after incubation,
isolating EV-derived small RNAs,
encapsulating EV-derived small RNAs in cationic liposomes,
characterizing by comprising the steps of obtaining the resulting encapsulated small RNAs and cell suspension extract as the raw material.

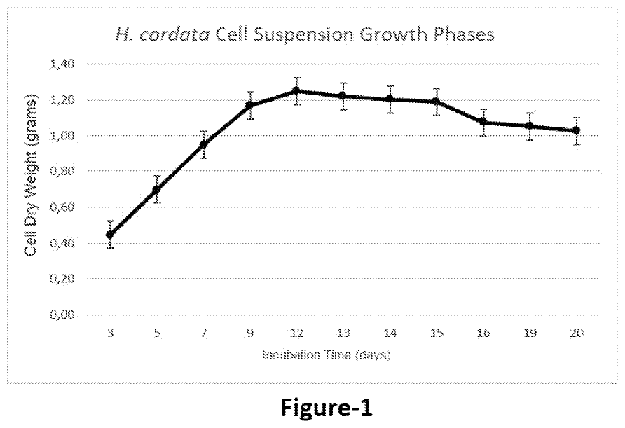

FIG. 1 presents the growth phases graph of H. cordata cell suspension,

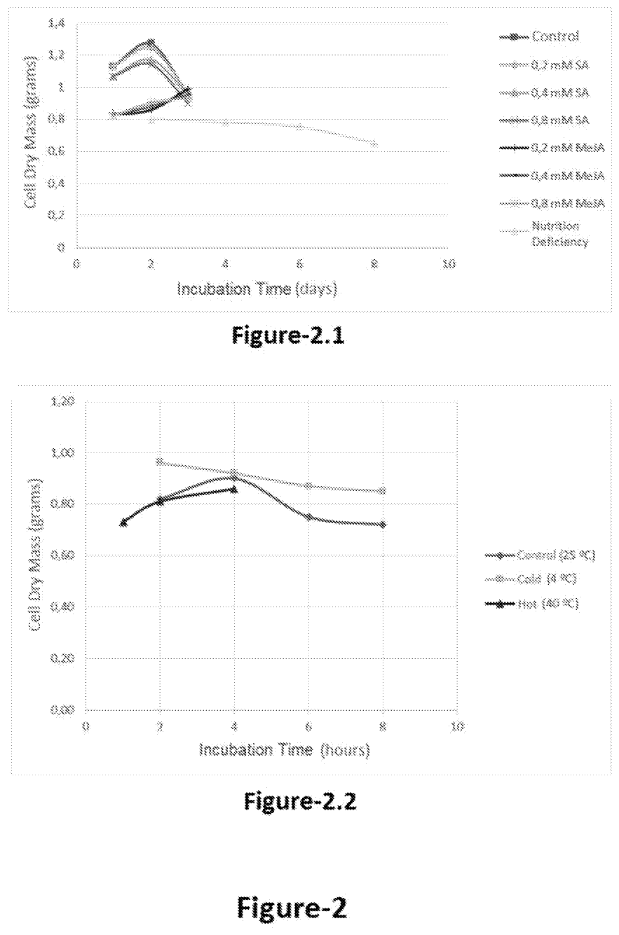

In FIG. 2, the evaluation of the effect of elicitor applications on H. cordata cell suspension biomass is shown,

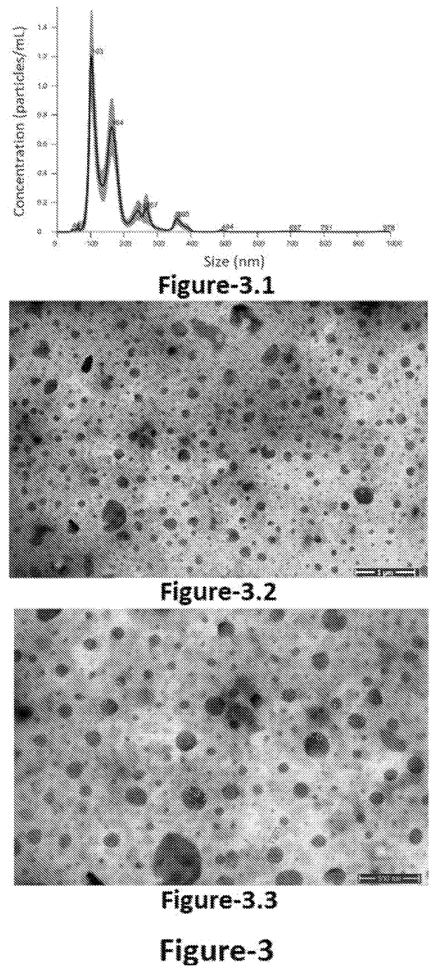

FIG. 3 displays NTA measurement results and S-TEM images of H. cordata cell suspension culture-derived EVs,

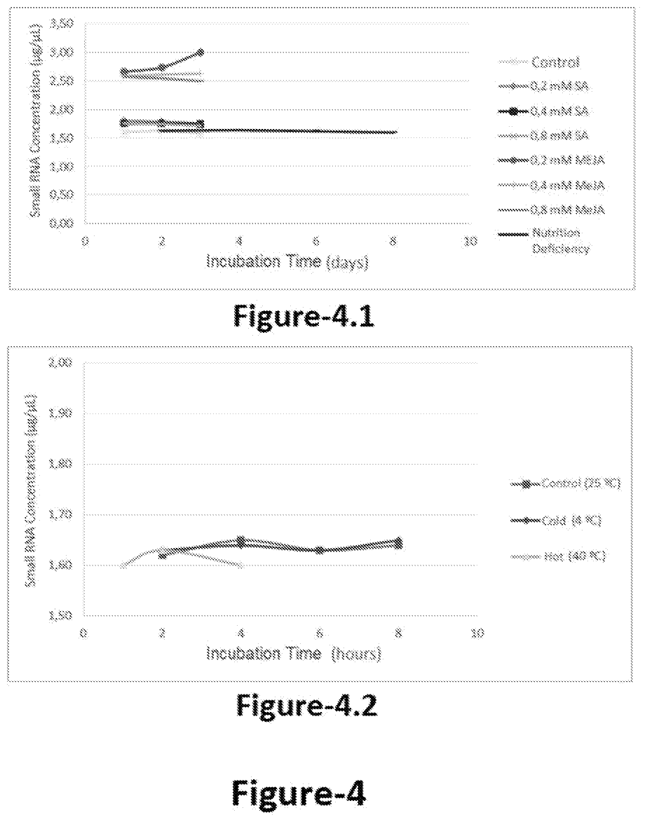

In FIG. 4, the evaluation of the effect of elicitor applications on the amount of small RNAs obtained from H. cordata cell suspension EVs is demonstrated,

FIG. 5 shows the results obtained from the Small RNA-Sequencing reaction,

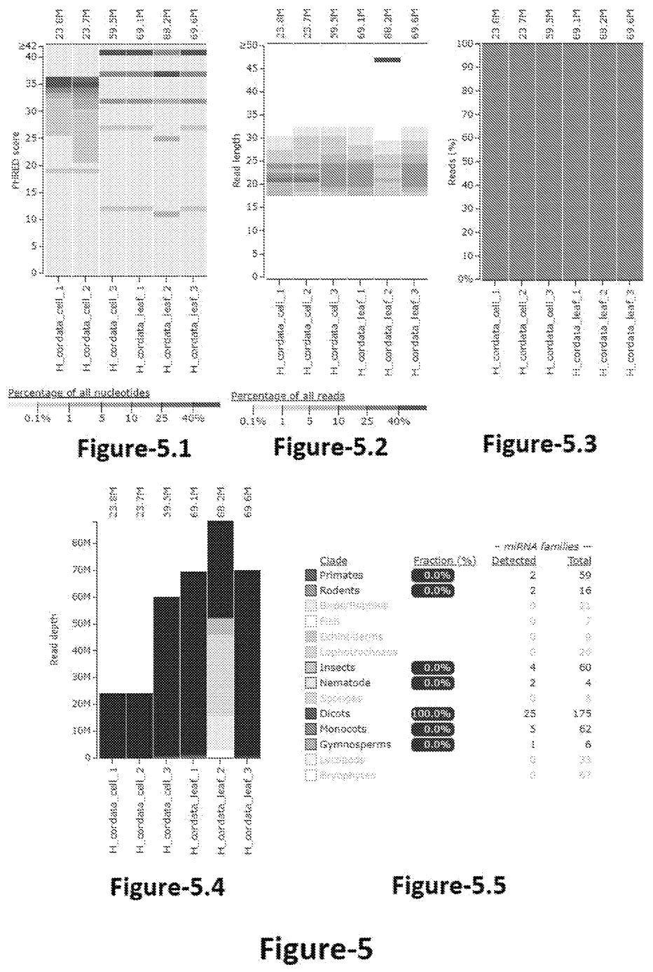

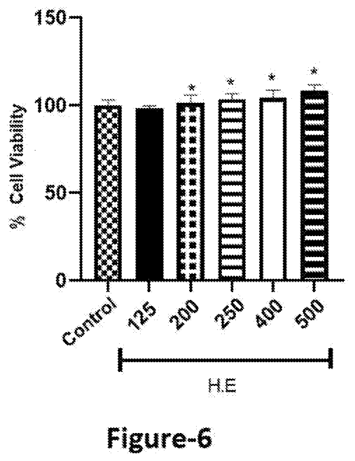

FIG. 6 depicts the effect of H. cordata cell suspension extract on cell viability,

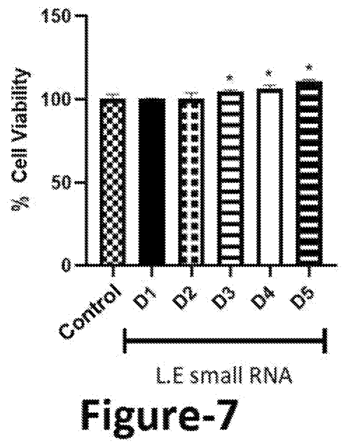

FIG. 7 demonstrates the effect of liposome-encapsulated small RNAs on cell viability,

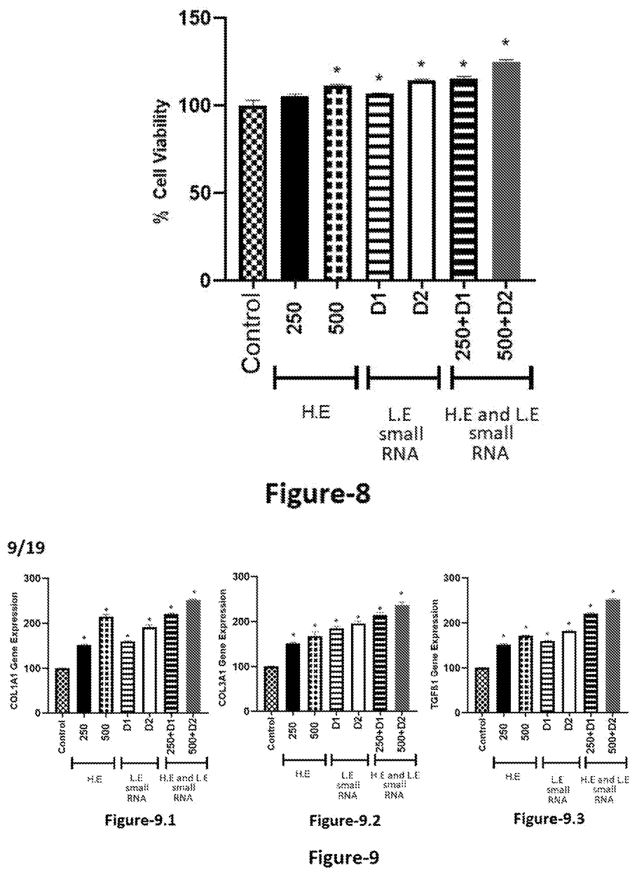

FIG. 8 presents the effect of the combination of H. cordata cell suspension extract and liposome-encapsulated small RNA composition on cell viability,

In FIG. 9, the effect of H.E, L.E small RNAs, and H.E and L.E combined small RNA applications on the expression of genes related to anti-aging activity is shown,

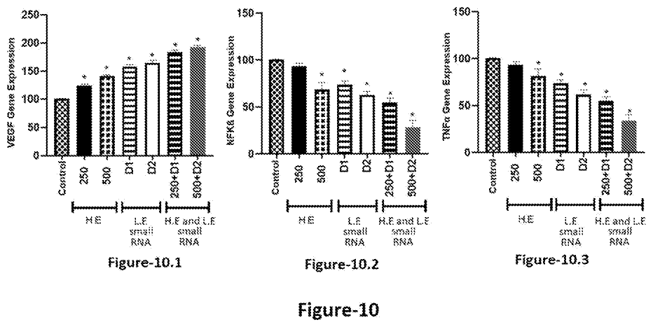

FIG. 10 demonstrates the impact of H.E, L.E small RNAs, and the combined application of H.E and L.E small RNAs on the expression of genes associated with wound healing,

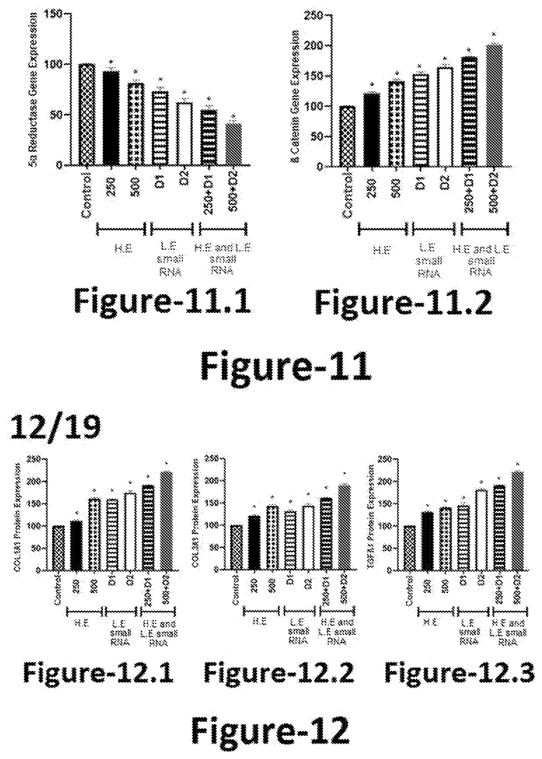

FIG. 11 depicts the impact of applying H.E, L.E small RNAs, and a combination of H.E and L.E small RNAs on the expression of genes associated with preventing hair loss,

FIG. 12 depicts the effect of H.E, L.E small RNAs, and H.E and L.E combined small RNA applications on the expression of anti-aging activity proteins,

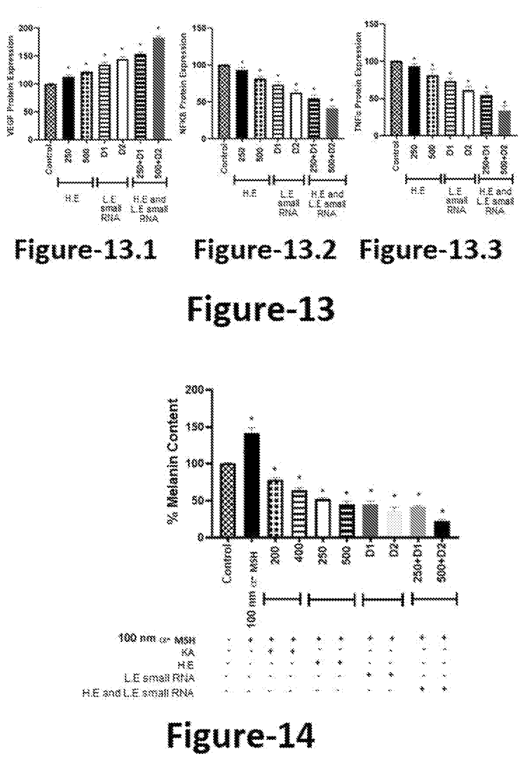

FIG. 13 demonstrates the effect of H.E, L.E small RNAs, and H.E and L.E combined small RNA applications on the expression of proteins related to wound healing,

FIG. 14 shows the effect of H.E, L.E small RNAs, and H.E and L.E combined small RNA applications on melanin content,

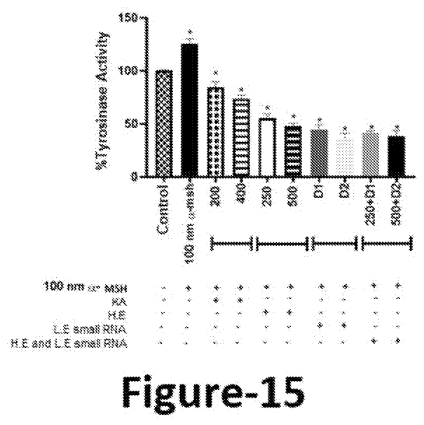

FIG. 15 demonstrates the effect of H.E, L.E small RNAs, and H.E and L.E combined small RNA applications on tyrosinase activity,

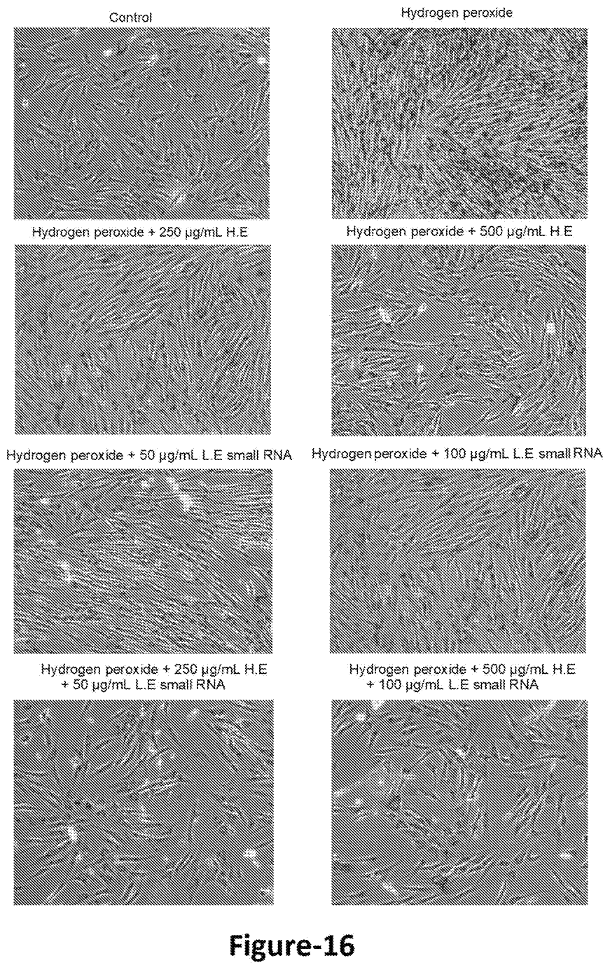

FIG. 16 depicts the effect of H.E, L.E small RNAs, and H.E and L.E combined small RNA applications on cell senescence,

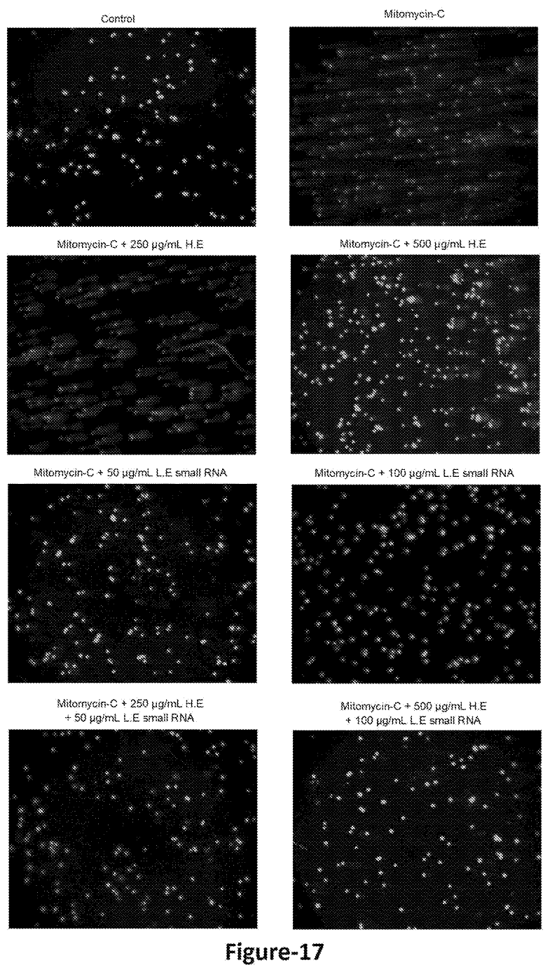

FIG. 17 shows the effect of H.E, L.E small RNAs, and H.E and L.E combined small RNA applications on protective effects against DNA damage,

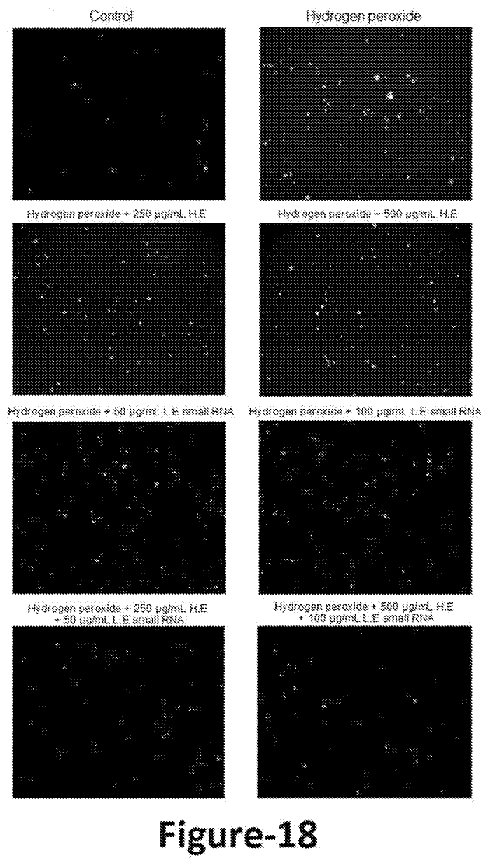

FIG. 18 demonstrates the effect of H.E, L.E small RNAs, and H.E and L.E combined small RNA applications on the formation of intracellular reactive oxygen species (ROS),

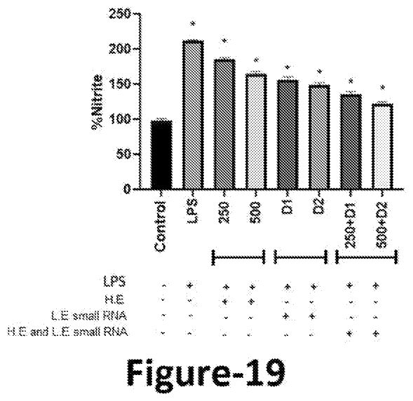

FIG. 19 indicates the effect of H.E, L.E small RNAs, and H.E and L.E combined small RNA applications on inflammation-induced nitrite formation.

1. Obtaining H. cordata Seedlings

H. cordata seedlings were obtained to compare the yield and composition of small RNAs derived from H. cordata adult leaf tissue and cell suspension culture medium EVs. H. cordata seeds were stored at 2-8° C. The seeds were placed in a moist, soft-tissue medium with a photoperiod of 16 hours light/8 hours dark at 25° C. Germination occurred after 15 days, and complete plants were obtained after 1 month.

2. Obtaining H. cordata Cell Suspension Cultures

H. cordata seeds were surface-sterilized by soaking in a 15% NaOCl solution for 15 minutes, followed by three rinses with distilled water. The sterilized seeds were placed in an in vitro culture medium containing preferably 1 mg/L BAP, 30 g/L sugar, and 4 g/L agar, adjusted to pH 5.8 (Table 1). The seeds were then incubated at 22±2° C. with a photoperiod of 16 hours light/8 hours dark in a climate chamber for conditioning. In vitro seedlings were obtained in approximately 14-20 days. To propagate the in vitro seedlings, subculture was performed by transferring them to the fresh culture medium at 3-4 week intervals. For callus induction, leaf and petiole explants of in vitro seedlings were wounded under aseptic conditions using a scalpel and placed in Gamborg's B5 medium containing 1 mg/L 2,4-D and 0.5 mg/L kinetin (Table 2). Leaf and petiole explants were used for callus induction in this application. Alternatively, root or stem explants of the plant can also be used. The explants were incubated in the dark at 25° C. until callus formation was observed. The explants were transferred to a fresh culture medium at 4-week intervals. When a sufficient amount of fragile and suspensible callus was obtained, the suspension culture stage was initiated. The culture medium with the most effective callus induction was used for the cell suspension culture. For the cell suspension culture, preferably 4-4.5 g of 0-45 day-old callus derived from H. cordata explants was transferred to 250 mL Erlenmeyer flasks containing 50 mL of liquid culture medium, and the flasks were incubated at 20-25° C. and 110 rpm in the dark until a suspension consisting of separated single or a few cell groups was obtained. Preferably, after approximately 4 weeks, the suspension containing large callus masses was passed through a sterile sieve (30 mesh) to remove them. The sieved suspension was subcultured by adding fresh culture medium in a 1:3 ratio. The Erlenmeyer flasks were then continued to be incubated at 110 rpm. The growth parameters of the cells in the generated suspension (cell count, pellet cell volume (PCV), settled cell volume (SCV), and cell viability) were determined weekly. The cell suspensions were subcultured at intervals of 2-3 weeks to increase the suspension volume.

TABLE 1
The quantities of macro- and micro-elements and
vitamins present in the MS nutrient medium.

| Macro Elements | mg/L |
|---|---|
| CaCl₂ | 332.02 |
| KH₂PO₄ | 170.00 |
| KNO₃ | 1900.54 |
| MgSO₄ | 180.54 |
| NH₄NO₃ | 1650.00 |
| Micro Elements | mg/L |
| CoCl₂•6H₂O | 0.025 |
| CuSO₄•5H₂O | 0.025 |
| FeNaEDTA | 36.70 |
| H₃BO₃ | 6.20 |
| Kl | 0.83 |
| MnSO₄•H₂O | 16.90 |
| Na₂MoO₄•2H₂O | 0.25 |
| ZnSO₄•7H₂O | 8.60 |
| Vitamins | mg/L |
| Glycine | 2.00 |
| Myo-inositol | 100.00 |
| Nicotinic acid | 0.50 |
| Pyridoxine HCl | 0.50 |
| Thiamine HCl | 0.10 |

TABLE 2
The quantities of macro and micro elements and
vitamins present in the Gamborg's B5 nutrient medium.

| Macro Elements | mg/L |
|---|---|
| CaCl₂ | 113.23 |
| NaH₂PO₄ | 130.44 |
| KNO₃ | 2500.00 |
| MgSO₄ | 121.56 |
| (NH₄)2SO₄ | 134.00 |
| Micro Elements | mg/L |
| CoCl₂•6H₂O | 0.025 |
| CuSO₄•5H₂O | 0.025 |
| FeNaEDTA | 36.70 |
| H₃BO₃ | 3.00 |
| Kl | 0.75 |
| MnSO₄•H₂O | 10.00 |
| Na₂MoO₄•2H₂O | 0.25 |
| ZnSO₄•7H₂O | 2.00 |
| Vitamins | mg/L |
| Myo-inositol | 100.00 |
| Nicotinic acid | 1.00 |
| Pyridoxine HCl | 1.00 |
| Thiamine HCl | 10.00 |

3. The Determination of Growth Phases of H. cordata Cell Suspension

To determine the growth phases of H. cordata suspension cells, these cells were transferred onto filter paper through a Büchner funnel and Nuche flask under aseptic conditions, separating them from the nutrient medium. For the 20-day experiment, a total of 22 Erlenmeyers with a volume of 250 mL were each inoculated with 4 grams of fresh cells in 50 mL of nutrient medium. These 22 flasks were incubated at 25° C. on an orbital shaker at 110 rpm for harvesting on different days. To determine the growth phases, every 2 days, two flasks were filtered, the cells' fresh weights were measured, and after lyophilization, their dry weights were recorded. The growth phases of H. cordata suspension cells were determined using the 20-day fresh/dry weight data. After lyophilization, the cells were stored at 2-8° C. for further analysis.

Based on the lyophilized H. cordata cell weights, the growth phase curve was established, where the culture's 0-3 days represented the lag phase, 3-12 days represented the logarithmic phase, 12-15 days represented the stationary phase, and the period from the 16th day onwards represented the decline phase (FIG. 1).

### 4. The Treatments of Elicitor

In previous studies, it has been reported that nutrient deficiency, heat, cold, salicylic acid (SA), and methyl jasmonate (MeJA) applications increase small RNA synthesis in plants (Zeng et al., 2014, ‘Role of microRNAs in plant responses to nutrient stress’, Plant and Soil, 374(1-2), pp. 1005-1021; Lv et al., 2010, ‘Profiling of cold-stress-responsive miRNAs in rice by microarrays’, Gene, 459(1-2), pp. 39-47; Zhang et al., 2009, ‘Deep sequencing of Brachypodium small RNAs at the global genome level identifies microRNAs involved in cold stress response’, BMC Genomics, 10(1), p. 449; Lu et al., 2008, ‘Stress-responsive microRNAs in Populus’, The Plant Journal: For Cell and Molecular Biology, 55(1), pp. 131-151; Xin et al., 2011, ‘Identification and characterization of wheat long non-protein coding RNAs responsive to powdery mildew infection and heat stress by using microarray analysis and SBS sequencing’, BMC Plant Biology, 11(1), p. 61; Zhang et al., 2022, ‘Roles of microRNAs in abiotic stress response and characteristics regulation of plant’, Frontiers in Plant Science, 13; Rutter et al., 2017, ‘Extracellular Vesicles Isolated from the Leaf Apoplast Carry Stress-Response Proteins1[OPEN]’, Plant Physiology, 173(1), pp. 728-741). To obtain efficient small RNA synthesis in H. cordata cell suspensions, various elicitor treatments were performed, including nutrient limitation, cold and heat incubation, and incubation with salicylic acid and MeJA.

### a) Exposure to Nutrient Deficiency

H. cordata cell suspension cultures were subjected to additional incubation in the same nutrient medium for 2, 4, 6, and 8 days, in addition to the standard subculture period of 14 days.

### b) Cold and Heat Incubation

On the 14th day of the culture, the cell suspension was incubated at 4° C. for 6, 8, 12, and 24 hours for cold treatment and at 40° C. for 1, 2, and 4 hours for heat treatment.

### c) MeJA and SA Treatment

On the 14th day of the culture, the cell suspension medium was supplemented with 0.2-0.4 and 0.8 mM MeJA or SA, and the suspensions were subjected to additional incubation for 1, 2, and 3 days.

5. Extraction of H. cordata Cell Suspensions After Elicitation

After the incubation periods of H. cordata cell suspension cultures were completed, the cells were passed through a filter, and the cultures were terminated. Subsequently, cells from all groups were lyophilized. The weights of the lyophilized cells were measured and recorded. No statistically significant difference in the quantity of H. cordata cell suspensions subjected to salicylic acid, MeJA, cold, and heat treatments was found compared to the control group. However, cells exposed to nutrient deficiency for 6 and 8 days exhibited a respective decrease in quantity by 1.1 and 1.3 times (FIGS. 2A and 2B).

Lyophilized cells were stored at −80° C. to be used for the preparation of the necessary extracts for in vitro experiments. Only lyophilized cells from the elicitation with the most efficient small RNA from EVs were subjected to extraction. For extraction, lyophilized H. cordata cells were homogenized in 70% ethanol at a ratio of 1:40 (w/v) using a rotor-stator homogenizer for 15 minutes. Then, the homogenates were extracted on a shaker at 110 rpm for 24 hours at 22±2° C. After completion of incubation, cell aggregates were separated using a filter. The filtered nutrient medium was passed through a filter paper with a pore size of 4-7 microns and filtered again. The solvents were evaporated under low pressure at 40° C. using a rotary evaporator to concentrate the extracts. The remaining solvents in the concentrated extracts were removed using a lyophilizer. The obtained H. cordata cell extract was used alone and/or in experiments with small RNA.

6. Isolation of EVs from H. cordata Adult Tissues and Cell Suspension Nutrient Media

EV isolation procedure was applied to both differentiated adult tissues (leaves) and suspension groups with or without elicitation in their respective nutrient media:

The isolation of EVs from H. cordata leaves and cell suspension culture nutrient media was carried out using the method developed by Huan et al. (2021 ‘Effective methods for isolation and purification of extracellular vesicles from plants,’ Journal of Integrative Plant Biology, 63(12), pp. 2020-2030). The method for isolating plant EVs generally involves two steps: (i) obtaining leaf apoplastic fluid (AWS) through vacuum infiltration and collecting AWS using centrifugation, (ii) filtering the obtained AWS to isolate plant EVs using sequential centrifugation. EV isolation from mature leaf tissue was performed after obtaining AWS, while from cell suspension culture, EVs were obtained directly from the culture medium. First, the base of mature leaves was separated from the stem using a sterile scalpel to minimize potential contaminants from the vascular bundle. Approximately 20-45 plant leaves were used for each experiment, and the total leaf amount was recorded using a precision balance. After washing the plant leaves, they were carefully placed inside a 200 mL syringe and gently vacuum-infiltrated with infiltration buffer [20 mmol/L L2-[N-morpholino] ethanesulfonic acid (MES) hydrate, 2 mmol/L CaCl₂, 0.1 mol/L NaCl, pH 6.0] for 20-30 seconds. Excess infiltration buffer on the leaf surface was removed using drying paper, and the leaves were fixed on a short plastic stick. The apex of the leaves was positioned upwards and placed into a 50 mL centrifuge tube, then centrifuged at 4° C. and 1,000 g for 15 minutes. After centrifugation, the isolation of EVs from AWS required the removal of relatively large-sized cellular debris. At this stage, the AWS solution was centrifuged at 4° C. and 2,000 g for 10 minutes and then passed through 0.45 μm pore-sized filters. The clear supernatant obtained after filtration was used for EV isolation. The supernatant from the conical centrifuge tube was transferred to ultracentrifuge tubes using a sterile pipette and centrifuged at 4° C. and 10,000 g for 45 minutes. After removing the pellet, the supernatant was further centrifuged at 4° C. and 100,000 g for 1 hour. The resulting pellet was washed with 10 mL infiltration buffer and centrifuged at the same speed and duration. The EVs were finally isolated for RNA extraction and stored at −86° C. in an ultra-deep freezer. All these procedures were performed for mature leaf tissue and cell suspension culture nutrient media. The main difference in EV isolation was that, for cell suspension culture, EV isolation was directly performed from the culture medium without AWS isolation. In conclusion, the size and quantitative measurements of the obtained EVs were carried out.

The concentration and size of the obtained EVs were determined using a nanoparticle tracking device (NTA, NS300, Nanosight). According to the NTA analysis results, the concentration of EVs obtained from H. cordata cell suspension treated with the most effective elicitor application (0.2 mM MeJA with 72 hours of incubation) was determined to be 7.4×10⁹ particles/mL in the nutrient medium (FIG. 3A). The imaging of EVs was conducted using a scanning transmission electron microscope (S-TEM, Quattro S, Thermo Scientific) with an acceleration voltage of 30 kV. The analyzed EVs were found to have sizes ranging from 120 to 150 nm (FIG. 3B).

a) the Isolation of Small RNAs from EVs Obtained from Both H. cordata Adult Tissues and Cell Suspension Nutrient Medium

After the isolation of EVs, to prevent exogenous small RNA contamination, the isolated EVs were treated with Micrococcal nuclease, an endo-exonuclease that cleaves single-stranded RNAs, thereby removing non-EV-derived small RNAs. For this reaction, 10 units of MNase (Thermo Fisher) were added to the isolated EVs and incubated at 37° C. for 15 minutes. To obtain sufficient EVs for total RNA isolation, EVs isolated in four separate tubes were combined. Samples belonging to each group (adult leaf tissue and cell suspension nutrient medium-derived) were pooled separately. For total RNA isolation, the TRIzol™ Reagent (Thermo Fisher Scientific) kit was used (Cai et al., 2018, “Plants send small RNAs in extracellular vesicles to fungal pathogen to silence virulence genes,” Science, volume 360(6393), pages 1126-1129). One milliliter of TRIzol™ Reagent was added to the EV samples and homogenized at 4,000 rpm for 60 seconds using a MagNA Lyser (Roche, Germany) device without delay. Following the kit protocol, the completely homogenized samples were incubated at room temperature for 5 minutes and transferred to sterile clean 1.5 mL microcentrifuge tubes. Then, approximately 200 μL of chloroform was added. The homogenate was inverted to mix the contents and incubated at room temperature for about 5 minutes before being centrifuged at 4° C. and 12,000 g for 15 minutes. The transparent phase at the top formed after centrifugation was carefully transferred to a new 1.5 mL microcentrifuge tube without disturbing the other phases. 500 μL of isopropanol was added to this phase containing RNA, and the tube was inverted to mix the contents. After incubating at room temperature for about 10 minutes, the tubes were centrifuged at 4° C. and 12,000 g for 10 minutes. The resulting supernatant was separated from the RNA pellet at the bottom of the tube, and the RNA pellet was washed with 1 mL of 75% ethanol. The tubes were inverted, and the pellet was allowed to float in the ethanol. Then, it was centrifuged at 4° C. and 7,500 g for 5 minutes. To completely remove the alcohol in the tube, the caps were opened, and the tubes were placed upside down inside a laminar cabinet. The alcohol was removed entirely, and then 40 μL of ultra-pure water was added to dissolve the RNA pellet on a dry heating block at 37° C. The isolated RNA samples were incubated with DNase I (Thermo Scientific) enzyme at 37° C. for 15 minutes. Afterward, the RNA isolates were stored in a −86° C. deep freezer for spectrophotometric and fluorometric measurements. The RNA samples were measured using a NanoDrop 2000c (Thermo Scientific) spectrophotometer and a Qubit 4.0 (Life Science) fluorometer, and the measurement results were recorded.

Different elicitor applications were evaluated, and the amounts of isolated small RNAs from cell suspension EVs (extracellular vesicles) belonging to the control group were assessed. It was determined that SA (salicylic acid), cold, heat, and nutrient deficiency applications did not result in a significant increase in the amount of RNA obtained from H. cordata cell suspension nutrient medium EVs compared to the control group (p<0.05). However, it was observed that incubation of cells with different concentrations of MeJA (methyl jasmonate) led to an increase in the amount of RNA obtained from the nutrient medium EVs compared to the control group. Specifically, it was found that the amount of small RNA obtained from the group incubated with 0.2 mM MeJA for 72 hours was 1.9 times higher (3000 ng/μL) than that obtained from the control group. Considering this, small RNA with the highest yield, derived from cell suspension EVs incubated with 0.2 mM MeJA for 72 hours, was utilized for further studies involving liposome preparation and in vitro efficacy experiments (FIGS. 4A and 4B).

b) Examining the Small RNA Profile Obtained from H. cordata Adult Tissues and Cell Suspension Nutrient Medium EVs

The small RNA profiles of H. cordata leaf and cell suspension culture medium EVs were examined to demonstrate whether they differ using transcriptomic experimental approaches, and RNA-Sequencing (RNA-Seq) analyses were performed with the isolated RNAs. Firstly, 7 μL of nuclease-free water was used to dissolve the isolated RNAs, and RNA integrity was measured using the Bioanalyzer pico RNA chip (Agilent), followed by cDNA synthesis. The small RNA sequencing library preparation was performed according to the NEBNext® Small RNA Library Prep for Illumina kit protocol, as described in the Matera et al. (2022) study. Briefly, three μL of total RNA isolate was diluted 1:10 and mixed with 1 μL of the 3′ SR adapter at 70° C. for 2 minutes. Subsequently, in each tube, 6.5 μL of the 3′ ligation mixture (5 μL of 3′ ligation buffer and 1.5 μL of 3′ enzyme) was added and incubated at 25° C. for 1 hour. After incubation, 2.75 μL of the SR mixture (2.5 μL of nuclease-free water and 0.25 μL of SR RT primer) was added to each reaction and incubated at 75° C. for 5 minutes, 37° C. for 15 minutes, and 25° C. for 15 minutes. Pre-denatured and 1:10 diluted 5′ adapter with 5′ ligation mixture (0.5 μL of 5′ ligation reaction buffer, 1.25 μL of 5′ ligation enzyme) was added to each tube and incubated at 25° C. for 1 hour. For the first-strand cDNA synthesis, 5 μL of cDNA mixture was added to each tube and incubated at 50° C. for 1 hour, followed by 70° C. for 15 minutes to continue the incubation. The obtained cDNAs were used for the PCR reaction. A sterile 0.2 mL PCR tube was used to prepare the PCR mixture, which contained 10 μL of cDNA, NEB indexes, and SR primers, with a total reaction volume of 50 μL (25 μL of LongAmp Taq 2× mix, 12.5 μL of nuclease-free water, 1.25 μL of SR primer, 1.25 μL of index primer). The PCR reaction cycle was performed in a Thermal Cycler (BioRad) device as follows: initial denaturation at 94° C. for 30 seconds, 20 cycles of denaturation at 94° C. for 15 seconds, annealing at 62° C. for 30 seconds, and elongation at 70° C. for 30 seconds. After PCR, the obtained amplicons were purified with AMPure XL beads (Beckman Coulter), and the amplicon length was checked using the QIAxcel Advanced (Qiagen) device. Following library preparation, the products were subjected to single-end 50-base (SE50) mode sequencing on the Illumina NextSeq 500 platform. The sequencing reaction was outsourced, and the new generation sequencing reads in “.fastq” format were processed on our laboratory workstation.

### c) The Bioinformatics Analysis of Small RNAs

A next-generation sequencing approach was performed to experimentally determine whether the small RNA composition and dynamics differ in adult leaf tissue and cell suspension culture EVs (extracellular vesicles). Raw reads were obtained in “.fastq” format. The raw reads resulting from the sequencing were first subjected to the filtering process using the fastp tool v0.20.1 (Chen et al., 2018, “fastp: an ultra-fast all-in-one FASTQ preprocessor”, Bioinformatics (Oxford, England), 34(17), ss. i884-i890) to obtain high-quality clean reads. The quality control and filtering process included the following steps: (i) removal of reads with a Phred score of less than 20 (<Q20), (ii) removal of reads shorter than 30 nt (nucleotides) and longer than 18 nt, (iii) adapter trimming, and (iv) removal of reads containing unreadable nucleotides (“N”). The obtained high-quality reads were clustered using the “collapse_reads.pl” script within the mirDeep2 software (Friedländer et al., 2012, “miRDeep2 accurately identifies known and hundreds of novel microRNA genes in seven animal clades”, Nucleic Acids Research, 40(1), ss. 37-52) and aligned to the miRBase v22.1 database (Kozomara et al., 2019, “miRBase: from microRNA sequences to function”, Nucleic Acids Research, 47(D1), ss. D155-D162) containing plant microRNAs (miRNA) using BlastN (-task “blastn-short”) (e-value cutoff of 1e-10). This allowed the identification of known miRNA compositions within the EVs. Additionally, known and previously uncharacterized miRNAs (Novel) were identified using the miRDeep-P2 v.1.1.4 software (Kuang et al., 2019, “miRDeep-P2: accurate and fast analysis of the microRNA transcriptome in plants”, Bioinformatics (Oxford, England), 35(14), ss. 2521-2522). The miRNAs that showed statistically significant differential expression in the miRNA expression levels observed in adult leaf tissue and cell suspension EVs were determined using the DEUS (Jeske et al., 2019, “DEUS: an R package for accurate small RNA profiling based on differential expression of unique sequences”, Bioinformatics (Oxford, England), 35(22), ss. 4834-4836) and edgeR v3.30.3 (Robinson et al., 2010, “edgeR: a Bioconductor package for differential expression analysis of digital gene expression data”, Bioinformatics (Oxford, England), 26(1), ss. 139-140) software (with adjusted p-value ≤0.05 and log₂FC≥1.0). Other potential small RNA types present in the EVs, such as tRNA, rRNA, snoRNA, and other non-coding RNAs, were aligned to the Rfam v14.2 database (Kalvari et al., 2020, “Rfam 14: expanded coverage of metagenomic, viral and microRNA families”, Nucleic Acids Research, 49(D1), ss. D192-D200) using BlastN (-task “blastn-short”) and identified with an e-value cutoff of 1 e-10.

The main subject of our patent application is that the profile and dynamics of small RNAs, including microRNAs, obtained from H. cordata cell suspension culture medium and adult leaf tissues EVs (extracellular vesicles) are different. Thus, due to the distinct small RNA profiles present in EVs during early-adult developmental stages (cell suspension and mature leaf tissue), these small RNAs may exhibit varying potential to regulate gene expression in mammalian cells. In this regard, small RNA profiling was performed from 3 samples of H. cordata cell suspension and 3 samples of H. cordata adult leaf tissues EVs. All bioinformatics analyses were conducted on workstations available in our laboratory. The summary statistical data and information on the quality of the reads obtained from the small RNA sequencing reaction are provided below (Table 3, FIGS. 5.A, B, C, D, and E). For samples obtained from H. cordata cell suspension EVs, a total of ~106 million single-end reads and 2.39 billion bases (nucleotides) were obtained. In the adult leaf tissue group, ~226 million single-end reads and 6.34 billion bases (nucleotides) were obtained.

TABLE 3
Summary statistics for small RNA sequencing reaction from H. cordata
cell suspension and adult leaf tissue EVs.

| | Read | | Lenght | Q20 | Q30 | GC |
|---|---|---|---|---|---|---|
| Sample Name | Numbers | Total Bases | (bp) | (%) | (%) | (%) |
| H_cordata_ | 23,755,170 | 513,555,385 | 50 | 99.31 | 98.26 | 52.02 |
| cell_1 | | | | | | |
| H_cordata_ | 23,746,751 | 528,045,310 | 50 | 99.36 | 96.76 | 51.05 |
| cell_2 | | | | | | |
| H_cordata_ | 59,493,741 | 1.355,224,407 | 50 | 99.86 | 99.46 | 52.13 |
| cell_3 | | | | | | |
| H_cordata_ | 69,103,068 | 1.525,792,742 | 50 | 99.86 | 99.45 | 52.41 |
| leaf_1 | | | | | | |
| H_cordata_ | 88,233,225 | 3,236,138,778 | 50 | 98.93 | 96.71 | 47.12 |
| leaf_2 | | | | | | |
| H_cordata_ | 69,554,320 | 1.580,853,830 | 50 | 99.86 | 99.44 | 52.05 |
| leaf_3 | | | | | | |

The Phred Score (Q), which indicates the quality of the sequencing reads, is above 90% for all samples, indicating that high-quality reads suitable for analysis have been obtained. FIG. 5 shows the clustering of read lengths predominantly around 20-25 bases and the absence of adapter contamination. Furthermore, the read depth per sample above 20 million and the alignment of reads predominantly to dicot miRNAs suggest no contamination in our data.

To determine experimentally whether the small RNA profile and dynamics, including microRNAs, in EVs obtained from H. cordata cell suspension and adult leaf tissues are different, various differential expression analyses (DEG) were performed. The statistical significance of microRNA dynamics (up-regulated and down-regulated gene expression) in EVs obtained from H. cordata cell suspension and adult leaf tissues was assessed based on the following parameters: corrected P value <0.05 and log fold-change (log₂FC) >2 or log₂FC<−2. This analysis determined that in EVs from H. cordata cell suspension, compared to adult leaf tissue, the expression of 57 miRNAs increased and 9 miRNAs decreased (Table 4).

TABLE 4
Comparison of statistically significant expression levels of miRNAs
obtained from EVs derived from H. cordata cell suspension culture medium and
adult leaf tissue. (Hco: H. cordata).

| Expression | miRNA | padj | log2FoldChange | pvalue | stat | foldChange | log10padj |
|---|---|---|---|---|---|---|---|
| UP- | Hco- | 0.03232 | 13.08714 | 0.00307 | 0.03232 | 8702.07831 | 1.49059 |
| REGULATED- | miR169q | | | | | | |
| GENES | Hco- | 0.03507 | 12.80497 | 0.00402 | 0.03507 | 7156.13677 | 1.45502 |
| | miR169g | | | | | | |
| | Hco- | 0.01072 | 12.23012 | 0.00034 | 0.01072 | 4804.31571 | 1.96960 |
| | miR169k | | | | | | |
| | Hco- | 0.03757 | 12.15791 | 0.00566 | 0.03757 | 4569.76848 | 1.42521 |
| | miR5038b | | | | | | |
| | Hco- | 0.01072 | 12.11605 | 0.00036 | 0.01072 | 4439.08457 | 1.96960 |
| | miR408c | | | | | | |
| | Hco- | 0.00983 | 11.99731 | 0.00021 | 0.00983 | 4088.37119 | 2.00734 |
| | miR169r | | | | | | |
| | Hco- | 0.00983 | 11.49864 | 0.00018 | 0.00983 | 2893.58951 | 2.00734 |
| | miR5678 | | | | | | |
| | Hco- | 0.03232 | 11.14865 | 0.00312 | 0.03232 | 2270.27101 | 1.49059 |
| | miR156i | | | | | | |
| | Hco- | 0.04379 | 10.93896 | 0.00755 | 0.04379 | 1963.15089 | 1.35858 |
| | miR169o | | | | | | |
| | Hco- | 0.03634 | 10.62350 | 0.00482 | 0.03634 | 1577.58134 | 1.43962 |
| | miR5036a | | | | | | |
| | Hco- | 0.01192 | 10.26593 | 0.00063 | 0.01192 | 1231.26532 | 1.92374 |
| | miR408d | | | | | | |
| | Hco- | 0.01072 | 10.24288 | 0.00029 | 0.01072 | 1211.75099 | 1.96960 |
| | miR164i | | | | | | |
| | Hco- | 0.01141 | 10.15266 | 0.00055 | 0.01141 | 1138.29361 | 1.94253 |
| | miR171s | | | | | | |
| | Hco- | 0.04379 | 10.09937 | 0.00742 | 0.04379 | 1097.01997 | 1.35858 |
| | miR162b | | | | | | |
| | Hco- | 0.00347 | 9.98197 | 0.00005 | 0.00347 | 1011.28533 | 2.46019 |
| | miR396c | | | | | | |
| | Hco- | 0.01072 | 9.81827 | 0.00039 | 0.01072 | 902.80727 | 1.96960 |
| | miR399j | | | | | | |
| | Hco- | 0.02118 | 9.71939 | 0.00135 | 0.02118 | 842.99919 | 1.67418 |
| | miR10440 | | | | | | |
| | Hco- | 0.01072 | 9.62452 | 0.00040 | 0.01072 | 789.34925 | 1.96960 |
| | miR160i | | | | | | |
| | Hco- | 0.00983 | 9.57184 | 0.00022 | 0.00983 | 761.04346 | 2.00734 |
| | miR165 | | | | | | |
| | Hco- | 0.01141 | 9.05225 | 0.00054 | 0.01141 | 530.88406 | 1.94253 |
| | miR1511 | | | | | | |
| | Hco- | 0.03757 | 8.63859 | 0.00585 | 0.03757 | 398.54216 | 1.42521 |
| | miR399d | | | | | | |
| | Hco- | 0.03232 | 8.42464 | 0.00243 | 0.03232 | 343.61396 | 1.49059 |
| | miR399q | | | | | | |
| | Hco- | 0.01141 | 8.39585 | 0.00056 | 0.01141 | 336.82362 | 1.94253 |
| | miR319c | | | | | | |
| | Hco- | 0.01072 | 8.25402 | 0.00041 | 0.01072 | 305.28732 | 1.96960 |
| | miR169a | | | | | | |
| | Hco- | 0.02837 | 7.98962 | 0.00196 | 0.02837 | 254.16415 | 1.54709 |
| | miR399g | | | | | | |
| | Hco- | 0.03232 | 7.93508 | 0.00269 | 0.03232 | 244.73548 | 1.49059 |
| | miR4413b | | | | | | |
| | Hco- | 0.02129 | 7.88409 | 0.00141 | 0.02129 | 236.23673 | 1.67191 |
| | miR160d | | | | | | |
| | Hco- | 0.01769 | 7.80349 | 0.00103 | 0.01769 | 223.40050 | 1.75235 |
| | miR395k | | | | | | |
| | Hco- | 0.03232 | 7.67708 | 0.00254 | 0.03232 | 204.65931 | 1.49059 |
| | miR171f | | | | | | |
| | Hco- | 0.03232 | 7.53734 | 0.00295 | 0.03232 | 185.76569 | 1.49059 |
| | miRN1346 | | | | | | |
| | Hco- | 0.04660 | 7.53439 | 0.00824 | 0.04660 | 185.38614 | 1.33160 |
| | miR4416b | | | | | | |
| | Hco- | 0.01141 | 7.27939 | 0.00057 | 0.01141 | 155.35087 | 1.94253 |
| | miR171q | | | | | | |
| | Hco- | 0.03232 | 7.12104 | 0.00260 | 0.03232 | 139.20202 | 1.49059 |
| | miR5669 | | | | | | |
| | Hco- | 0.03507 | 6.65820 | 0.00426 | 0.03507 | 100.99903 | 1.45502 |
| | miR9750 | | | | | | |
| | Hco- | 0.03518 | 6.38947 | 0.00448 | 0.03518 | 83.83457 | 1.45373 |
| | miR4414 | | | | | | |
| | Hco- | 0.03318 | 5.85721 | 0.00348 | 0.03318 | 57.96896 | 1.47915 |
| | miR1510a | | | | | | |
| | Hco- | 0.04819 | 5.84968 | 0.00865 | 0.04819 | 57.66723 | 1.31709 |
| | miR1515b | | | | | | |
| | Hco- | 0.01769 | 5.71967 | 0.00098 | 0.01769 | 52.69762 | 1.75235 |
| | miR156f | | | | | | |
| | Hco- | 0.03232 | 5.71234 | 0.00290 | 0.03232 | 52.43074 | 1.49059 |
| | miR164e | | | | | | |
| | Hco- | 0.03232 | 5.39060 | 0.00280 | 0.03232 | 41.94996 | 1.49059 |
| | miR159a | | | | | | |
| | Hco- | 0.03208 | 5.30290 | 0.00230 | 0.03208 | 39.47600 | 1.49374 |
| | miR5786a | | | | | | |
| | Hco- | 0.03507 | 5.28559 | 0.00419 | 0.03507 | 39.00502 | 1.45502 |
| | miR167g | | | | | | |
| | Hco- | 0.03352 | 5.23130 | 0.00370 | 0.03352 | 37.56455 | 1.47464 |
| | miR319i | | | | | | |
| | Hco- | 0.03507 | 5.21108 | 0.00423 | 0.03507 | 37.04176 | 1.45502 |
| | miR397b | | | | | | |
| | Hco- | 0.03692 | 5.18563 | 0.00510 | 0.03692 | 36.39396 | 1.43273 |
| | miR1560 | | | | | | |
| | Hco- | 0.03290 | 5.08811 | 0.00336 | 0.03290 | 34.01528 | 1.48284 |
| | miR171m | | | | | | |
| | Hco- | 0.03776 | 4.99972 | 0.00605 | 0.03776 | 31.99390 | 1.42296 |
| | miR160b | | | | | | |
| | Hco- | 0.03757 | 4.88918 | 0.00574 | 0.03757 | 29.63391 | 1.42521 |
| | miR408a | | | | | | |
| | Hco- | 0.02108 | 4.82142 | 0.00128 | 0.02108 | 28.27437 | 1.67607 |
| | miR5781 | | | | | | |
| | Hco- | 0.03757 | 4.77008 | 0.00591 | 0.03757 | 27.28582 | 1.42521 |
| | miR319l | | | | | | |
| | Hco- | 0.04379 | 4.75201 | 0.00762 | 0.04379 | 26.94612 | 1.35858 |
| | miR1508a | | | | | | |
| | Hco- | 0.03782 | 4.73735 | 0.00616 | 0.03782 | 26.67384 | 1.42223 |
| | miR319h | | | | | | |
| | Hco- | 0.03757 | 4.73014 | 0.00566 | 0.03757 | 26.54078 | 1.42521 |
| | miR159g | | | | | | |
| | Hco- | 0.03757 | 4.68468 | 0.00585 | 0.03757 | 25.71761 | 1.42521 |
| | miR2606b | | | | | | |
| | Hco- | 0.03757 | 4.67983 | 0.00555 | 0.03757 | 25.63119 | 1.42521 |
| | miR159i | | | | | | |
| | Hco- | 0.03692 | 4.40048 | 0.00503 | 0.03692 | 21.11914 | 1.43273 |
| | miRN1286 | | | | | | |
| | Hco- | 0.04950 | 4.37768 | 0.00903 | 0.04950 | 20.78802 | 1.30535 |
| | miR171c | | | | | | |
| DOWN- | Hco- | 0.00001 | −11.04041 | 0.00000 | 0.00001 | 0.00047 | 4.95232 |
| REGULATED- | miR157g | | | | | | |
| GENES | Hco- | 0.00219 | −10.43091 | 0.00002 | 0.00219 | 0.00072 | 2.66008 |
| | miR395b | | | | | | |
| | Hco- | 0.00219 | −10.49049 | 0.00002 | 0.00219 | 0.00070 | 2.66008 |
| | miR390g | | | | | | |
| | Hco- | 0.00219 | −10.59533 | 0.00002 | 0.00219 | 0.00065 | 2.66008 |
| | miRN1303 | | | | | | |
| | Hco- | 0.03290 | −5.29839 | 0.00329 | 0.03290 | 0.02541 | 1.48284 |
| | miR319b | | | | | | |
| | Hco- | 0.03352 | −4.17374 | 0.00363 | 0.03352 | 0.05541 | 1.47464 |
| | miR394d | | | | | | |
| | Hco- | 0.03518 | −5.81034 | 0.00457 | 0.03518 | 0.01782 | 1.45373 |
| | miR1535 | | | | | | |
| | Hco- | 0.03518 | −8.86091 | 0.00446 | 0.03518 | 0.00215 | 1.45373 |
| | miR398b | | | | | | |
| | Hco- | 0.04206 | −7.14815 | 0.00697 | 0.04206 | 0.00705 | 1.37617 |
| | miR1711 | | | | | | |

According to the gene expression analysis, it is observed that the expression levels of microRNAs in EVs derived from H. cordata cell suspension culture medium and adult leaf tissues are different. Therefore, the distinct composition of small RNA types, such as miRNAs, in the cell suspension culture medium suggests that the miRNAs present in EVs from the cell suspension may have varying effects on target genes in the recipient cells.

### d) Encapsulation of Small RNAs

To ensure the stability of small RNAs, encapsulation within cationic liposomes was performed. The encapsulation process was carried out following the protocol used by Wu et al. (2011, “MicroRNA delivery by cationic lipoplexes for lung cancer therapy,” Molecular Pharmaceutics, 8(4), pp. 1381-1389). Empty liposomes were prepared by injecting the lipid mixture (DOTMA: cholesterol: TPGS=49.5:49.5:1) in ethanol into 20 mM HEPES buffer (pH=7.4) with a final concentration of 10:90 (ethanol/water). The small RNAs isolated from H. cordata cell suspension EVs (3 mg/mL) were added to the empty liposome mixture at a final concentration of 1 mg/mL, and the mixture was incubated at room temperature for 15 minutes. The liposome-encapsulated small RNAs were used in in vitro studies.

7. Extraction of H. cordata Cell Suspension and In Vitro Activity Tests of Small RNAs

### a) Effects on Cell Viability:

The effect of H. cordata cell suspension extract (H.E), small RNAs obtained from H. cordata cell suspension culture medium-derived extracellular vesicles (L.E small RNA), and the combined application of H.E and L.E small RNA on cell viability was determined on human normal fibroblast cells (CRL-2076). Cell culture incubation processes were conducted at 37° C. with 5% CO₂ in an incubator. Human fibroblast cells were seeded into 24-well plates at 1×10⁵ cells/well density. After 24 hours of incubation, the culture medium in the wells was removed, and the culture medium suspensions containing specific doses of the extract were added. After 48±0.5 hours of incubation with the extracts, the extract-culture medium solutions were removed, and MTT-culture medium suspensions with a final concentration of 5 mg/mL were added to the wells. The plates were left for 3±0.1 hours of incubation. At the end of the incubation period, the MTT-culture medium solution was removed, and DMSO was added to each well to dissolve the formazan salts produced by viable cells. The plates were shaken on a plate shaker for 15 minutes. Subsequently, the OD (optical density) values of the plates were read at 570 nm. Based on the obtained results, the viability levels of the cells exposed to specific doses of the substance were determined compared to the control cells. The experimental setup was designed to include wells with only a culture medium for blind testing, wells with control cells not exposed to any substance, and wells with cells exposed to various doses of the substance.

According to the in vitro cytotoxicity results, no significant changes were observed in H.E at doses of 125 and 200 μg/mL compared to the control. In contrast, at doses of 250, 400, and 500 μg/mL, an increase of 3%, 4%, 5%, and 9%, respectively, was determined (FIG. 6).

In the in vitro cytotoxicity experiment of L.E small RNA, it was found that concentrations of 25, 50, and 100 μg/mL increased cell viability by 4%, 6%, and 10%, respectively (FIG. 7).

In the combined application of H.E and L.E small RNA, cell viability increased by 15% at 250 μg/mL H.E+50 μg/mL L.E small RNA, and by 25% at 500 μg/mL H.E+100 μg/mL L.E small RNA, compared to the control group (FIG. 8).

Based on the in vitro cytotoxicity experiment results, the concentrations of 250 and 500 μg/mL for H.E and 50 and 100 μg/mL for L.E small RNA were used in the in vitro efficacy experiments.

### b) Determination of Anti-Aging, Wound Healing, and Anti-Hair Loss Activities at the Gene Level by RT-qPCR Analysis:

After seeding human fibroblast cells at a density of 5×10⁵ cells/flask in 25 cm² cell culture flasks, they were incubated for 24 hours. At the end of this period, the cells were treated with a serum-free medium containing the prepared extract and/or small RNA doses. After 48 hours of incubation, RNA was isolated from the cells in the flasks using an RNA isolation kit, and cDNA was synthesized from the obtained RNA. Subsequently, the effects of H. cordata cell suspension extract and small RNA on the expression of genes related to anti-aging, wound healing, and anti-inflammatory activity (COL1A1, COL3A1, TGFβ1, VEGF, NF-KB, and TNFα) and genes related to anti-hair loss activity (5α-Reductase and β-catenin) were analyzed using the SYBR green-based RT-qPCR method. The functions of the mentioned genes are as follows:

TGFβ1 (Transforming Growth Factor Beta 1): Stimulates fibroblasts and initiates the wound healing process by suppressing inflammation. It also orchestrates several cellular responses, such as neovascularization and extracellular matrix accumulation in tissue formation (Wang et al., 2013, “Effects of TRAP-1-like protein (TLP) gene on collagen synthesis induced by TGF-β/Smad signaling in human dermal fibroblasts”, PloS One, 8(2), s. e55899).

VEGF (Vascular Endothelial Growth Factor): Promotes epithelialization and collagen formation during wound healing (Wahyuningsih et al., 2019, “Ethanolic Extract Of Tithonia Diversifolia (Hemsley) A. Gray Inhibits Migration Activity And Decrease The Transforming Growth Factor-Beta1, Vegf Expression On Keloid Fibroblasts”, Asian Journal of Pharmaceutical and Clinical Research, 12(1), s. 342).

COL1A1 (Collagen Type 1 Alpha 1): Regulates the synthesis of collagen type 1 in various tissues, constituting approximately 70% of all collagen in the body. It is expressed throughout the wound-healing process (Inkinen, 2003, “Connective Tissue Formation in Wound Healing An experimental study”).

COL3A1 (Collagen Type 3 Alpha 1): Along with COL1A1, it contributes to wound healing in soft tissues, constituting 20-50% of adult collagen content (Inkinen, 2003, “Connective Tissue Formation in Wound Healing An experimental study”).

TNFα (Tumor Necrosis Factor Alpha): Suppresses collagen formation and stimulates collagenase synthesis (Chou et al., 1996, “TNF-alpha inactivation of collagen receptors: implications for fibroblast function and fibrosis”, Journal of Immunology (Baltimore, Md.: 1950), 156(11), ss. 4354-4362).

NF-KB (Nuclear Factor Kappa B): Involved in increasing cell proliferation and adhesion during wound healing and reducing the effects of inflammation and reactive oxygen derivatives (Park et al., 2018, “NF-κB signaling is key in the wound healing processes of silk fibroin”, Acta Biomaterialia, 67, ss. 183-195).

SRD5A2 (5α-Reductase): An enzyme that converts testosterone hormone into a more potent androgen called dihydrotestosterone hormone (DHT).

VEGF (Vascular Endothelial Growth Factor): Promotes the formation of capillaries that nourish the hair follicle.

β-catenin (CTNNB1): Participates in the Wnt-β-catenin signaling pathway, playing a role in hair follicle regeneration and the continuity of the anagen phase.

### RNA Isolation:

RNA isolation was performed according to the manufacturer's recommended protocol (E.Z.N.A. Total RNA Kit 1, Omega Bio-tek). Cells were removed from separate flasks using trypsin-EDTA and then centrifuged. After centrifugation, the obtained pellet was mixed with 700 μL of lysis buffer (TRK lysis buffer), and 20 μL of β-mercaptoethanol was added for every 1 mL of lysis buffer. The cell lysate was homogenized by placing it in a 1 mL centrifuge tube with beads and homogenizing it for 45 seconds using a magNa lyser (Roche) device. An equal volume of 70% ethanol was added to the lysate and carefully pipetted to mix. The samples were transferred to spin columns and centrifuged at 12,000 rpm for 30 seconds to allow the lysate to bind to the spin column while the flow-through was discarded. Then, 500 μL of wash solution 1 was added to the column, centrifuged at 12,000 rpm for 30 seconds, and the liquid collected at the bottom was carefully removed. The columns were washed twice with 500 μL of wash solution 2. For the drying step, the columns were transferred to a new collection tube and centrifuged at 14,000 rpm for 1 minute. For RNA elution, the columns were transferred to a 1.5 mL capped microcentrifuge tube, and 40 μL of water was added to the column, followed by centrifugation at 12,000 rpm for 2 minutes. This process allowed RNA molecules to be washed off the column filter and collected in the microcentrifuge tube with water.

cDNA Synthesis:

The RNA main stocks with predetermined quantities were diluted to 1000 ng using distilled water to prepare RNA intermediate stocks. The concentration of RNA intermediate stocks was reduced to 100 ng, and cDNA synthesis was performed using the cDNA kit (High-Capacity cDNA Reverse Transcription Kits, Applied Biosystems) following the recommended procedure by the manufacturer. The procedure for cDNA synthesis is listed step-by-step below:
1. Kit components were thawed on ice.
2. The cocktail provided in Table 5 was prepared on ice for each cDNA synthesis reaction.
3. Each cocktail was gently pipetted with 10 μL of 100 ng/μL RNA samples. The tubes were kept on ice until the volume and components were completed and transferred to the Thermal Cycler (T100, Biorad).
4. Finally, the tubes were incubated in the Thermal Cycler device at 25° C. for 10 minutes, 37° C. for 120 minutes, and 85° C. for 5 minutes to perform cDNA synthesis.

TABLE 5
The components of cDNA Synthesis Reaction

| Components | Quantities |
|---|---|
| 10X RT Buffer | 2 μL |
| 25X dNTPMix (100 mM) | 0.8 μL |
| 10X RT Random Primers | 2 μL |
| Multi Scribe Reverse | 1 μL |
| Transcriptase | |
| Nuclease-Free H₂O | 4.2 μL |
| Total cocktail volume | 10 μL |

5. The obtained cDNA main stocks were diluted 25 times and used for RT-qPCR studies. The expression levels of TGFβ1, VEGF, COL1A1, COL1A3, NF-KB, TNFα, SRD5A2, and CTNBB1 genes were determined using SYBR green method (PowerUp SYBR Green Master Mix, Applied Biosystems) with the primer pairs listed in Table 6.
6. The expression levels of the target genes were normalized to the reference gene's (ACTN) expression level, and fold changes were determined using the 2^{-ΔΔ} CT method (Schmittgen and Livak, 2008). The primer sequences used for gene expression analysis are provided in Table 6.

TABLE 6
Reverse and forward primer sequences of the
genes.

| Primers | Sequence |
|---|---|
| TGFβ1-Forward | AACCCACAACGAAATCTATGAC |
| TGFβ1-Reverse | TTCTCGGAGCTCTGATGTG |
| VEGF-Forward | GTATAAGTCCTGGAGCGTG |
| VEGF-Reverse | TTGTCACATCTGCAAGTACG |
| COL1A1-Forward | CTGGAAGAGTGGAGAGTACTG |
| COL1A1- Reverse | GTCTCCATGTTGCAGAAGAC |
| COL3A1-Forward | CAAGAGTGGAGAATACTGGGT |
| COL3A1- Reverse | CCCAGTTTCCATATTACAGAATACC |
| NF-KB-Forward | CCATCTATGACAGTAAAGCCC |
| NF-KB- Reverse | AATCCTTCCCAGACTCCAC |
| TNFα-Forward | CTCTAATCAGCCCTCTGGC |
| TNFα-Reverse | GAGGGTTTGCTACAACATGG |
| SRD5A2 Forward | ATGCTATCTTACGATTAC |
| SRD5A2 Reverse | TACCGATTACATACCATAGCT |
| CTNNB1 Forward | CATGTATTACATATATTTACGCT |
| CTNNB1 Reverse | TATCGATTACATGCTTCGATACG |
| ACTN Forward | CCAGAATCATGGTTTCCATAT |
| ACTN Reverse | ACTGCTAATACGATTACGTTA |

### Rt-qpcr Analysis:

The RT-qPCR cocktail used to determine the gene expression levels with SYBR Green method is shown in Table 7.

TABLE 7
The preparation of RT-qPCR cocktail.

| Component | Quantity |
|---|---|
| SYBR Green Master Mix | 5 μL |
| Forwad Primer | 0.5 μL |
| Reverse Primer | 0.5 μL |
| Nükleaz-free H₂O | 1.5 μL |
| Total cocktail volume | 7.5 μL |

1. The cocktail was distributed into the wells of the RT-qPCR 96-well microplate, with 7.5 μL in each well.
2. Then, cDNA samples were added to the wells, with 2.5 μL for each reaction, and the final volume was adjusted to 10 μL, with 3 technical replicates for each reaction.
3. The microplate was centrifuged at 1500 rpm for 2 minutes.
4. Finally, the reaction was carried out in the RT-qPCR instrument, following the thermal cycling conditions recommended by the SYBR Green manufacturer (Table 8).

TABLE 8
Thermal cycling conditions used in the RT-qPCR analysis.

| Deteksiyon | Syber Green | | |
|---|---|---|---|
| formatι | | | Analiz |
| Program adι | | Cycles | Modu |
| UDV Activation | | 1 | None |
| Dual-Lock DNA | | 1 | None |
| Polymerase | | | |
| Annealing | | 40 | Quantifi- |
| | | | cation |
| Melting | | 1 | Melting |
| | | | Curves |
| Cooling | | 1 | None |
| | | Hold | Ramp |
| Sιcaklιk Hedefleri | Acqusition | (hh:mm: | Rate(° C./ |
| Sιcaklιk (° C.) | Mode | ss) | s) |
| UDG Activation | | | |
| 50 | None | 00:02:00 | 4.4 |
| Dual-Lock DNA | | | |
| Polymerase | | | |
| 95 | None | 00:02:00 | 4.4 |
| Anneling | | | |
| 95 | None | 00:00:15 | 4.4 |
| 58 | None | 00:00:15 | 2.2 |
| 72 | Single | 00:01:00 | 4.4 |
| Melting | | | |
| | None | 00:00:15 | 1.6 |
| | None | 00:01:00 | 1.6 |
| | Continious | | 0.11 |
| Cooling | | | |
| 40 | None | 00:00:10 | 2.2 |

When the RT-qPCR analyses were evaluated, an increase in the expression of genes associated with anti-aging activity, namely COL1A1, COL3A1, and TGFβ1, was observed in response to H.E or L.E small RNA applications in a dose-dependent manner compared to the control group. The combined application of H.E and L.E small RNA resulted in the highest increase in the expression of these genes compared to other treatments (i.e., H.E or L.E small RNA alone) (FIGS. 9.A, B, and C).

Regarding the genes associated with wound healing, the expression of VEGF showed a dose-dependent increase in response to H.E application and L.E small RNA treatments compared to the control group. The combined application of H.E and L.E small RNA resulted in the highest dose-dependent increase in VEGF expression. On the other hand, the expression of NF-KB and TNFα, which are wound healing-related genes, decreased in response to H.E application in a dose-dependent manner. The expression of these genes was also dose-dependently reduced with L.E small RNA treatment compared to the control group, with the most significant reduction observed when H.E and L.E small RNA were applied together (FIGS. 10.A, B, and C).

As for the genes associated with anti-hair loss activity, the expression of 5 α-reductase decreased in response to H.E and L.E small RNA in a dose-dependent manner, with the most substantial reduction observed when H.E and L.E small RNA were applied together. The expression of β-catenin, a gene related to hair follicle stimulation mechanisms, increased in a dose-dependent manner with H.E and L.E small RNA treatments compared to the control group, and the highest level of expression was observed when H.E and L.E small RNA were applied together (FIGS. 11.A, B, and C).

The Anti-Aging Effects were Determined at the Protein Level Using the Western Blot Method:

Human normal fibroblast cells were seeded in 25 cm² cell culture flasks at a density of 5×10⁵ cells/flask and incubated for 24 hours. After incubation, the serum-containing medium was replaced with a serum-free medium, and H. cordata cell suspension extract and/or small RNA concentrations were applied. After 48 hours of incubation, protein isolation was performed from the cells on the plates using RIPA lysis buffer (RIPA Lysis Buffer system, Santa Cruz Biotechnology).

### The Protein Isolation Steps are as Follows:

1. For each 1 mL of RIPA Buffer, 10 μL of PMSF (phenylmethylsulfonyl fluoride), 10 μL of sodium orthovanadate solution, and 10-20 μL of protease inhibitor cocktail solution were added.
2. For protein isolation, the cells in the flask were washed with PBS, and then 700-800 μL of 0.25% Trypsin-EDTA was added to the cells, and the cells were incubated at 37¹C until they detached from the flask surface. The cells were collected in a centrifuge tube.
3. The tubes were centrifuged at 125 g for 5 minutes to pellet the cells.
4. After centrifugation, the supernatants were removed by vacuum aspiration. Then, the cells were washed with PBS and centrifuged again.
5. After centrifugation, the supernatants were removed again.
6. 1 mL of RIPA buffer was added to each tube, and the tubes were incubated on ice for 30 minutes.
7. After incubation, the samples were transferred to microcentrifuge tubes and centrifuged at 10,000 g for 10 minutes at 4° C.
8. The supernatants were transferred to new microcentrifuge tubes and labeled with the date and sample name.
   The Determination of Protein Concentration with BCA Kit:

The protein concentration was determined using the BCA kit. For this purpose, solutions were prepared according to the kit's recommendations (Table 9).

TABLE 9
Preparation of BSA Standards.

| | Volume to | Amount of | |
|---|---|---|---|
| Tube | Dilute | BSA to Add | Final Concentration |
| 1 | 0 | Stoktan 300 μL | 2000 μg/mL |
| 2 | 125 μL | Stoktan 375 μL | 1500 μg/mL |
| 3 | 325 μL | Stoktan 325 μL | 1000 μg/mL |
| 4 | 325 μL | Tüp 3′ten 325 | 500 μg/mL |
| | | μL alιnιr. | |
| 5 | 325 μL | Tüp 4′ten 325 | 250 μg/mL |
| | | μL alιnιr. | |
| 6 | 325 μL | Tüp 5′ten 325 | 125 μg/mL |
| | | μL alιnιr. | |
| 7 | 400 μL | Tüp 6′dan 100 | 25 μg/mL |
| | | μl alιnιr. | |
| 8 | 400 μL | 0 | 0 μg/mL = blank |
| Working | 50 units of | 1 unit of | 51 units of working |
| Solution | Solution A | Solution B | solution |

1. 25 μL of protein samples were added to microplate wells.
2. Each well was added with 200 μL of the working solution, and the plate was gently mixed.
3. Then, the plate was incubated at 37° C. for 30 minute s. After the incubation, the plate was left to cool at room temperature.
4. Finally, the plate was read at 562 nm using a microplate reader.
5. Using the measured absorbance values, a standard curve was created with the standards, and the protein concentrations in the samples were calculated based on the standard curve.

### Separation of Proteins by SDS-PAGE:

1. Cell lysates were mixed with 2× electrophoresis sample buffer (2× Sample
Buffer: 125 mM Tris pH 6.8, 4% SDS, 10% Glycerol, 0.006% Bromophenol Blue, 1.8% β-Mercaptoethanol) in equal volumes and denatured at 94° C. for 3-5 minutes.
2. 30 μg of protein was loaded into each well of the gel.
3. Samples mixed with Bromophenol Blue were run through the electrophoresis system until they reached the bottom of the gel.

### Protein Blotting:

Proteins were transferred onto a PVDF membrane using a semi-dry blotting device (Biorad-Transblot Turbo Transfer System).
1. PVDF membrane was soaked in methanol for 15 seconds.
2. The membrane was immersed in deionized water for 2 minutes.
3. The membrane was kept in a transfer buffer (25 mM Tris, 20% Methanol, pH 10.4) for 5 minutes.
4. One piece of filter paper was soaked in a transfer buffer and placed on the surface of the semi-dry blotting device.
5. The SDS-PAGE gel was placed on top of the filter paper, and another piece of filter paper was put on top of the gel.
6. The device was connected to a power supply, and the transfer was carried out.

### Blocking of the Membrane:

1. The membrane was removed from the blotting device and immersed in blocking buffer solution (5% non-fat dry milk, 10 mM Tris pH 7.5, 100 mM NaCl, 0.1% Tween 20).
2. The membrane was incubated in the blocking buffer at 37¹C for 30 minutes, at room temperature for 1 hour, or overnight at 4° C.

### Primary Antibody Application:

1. According to the manufacturer's recommendations, primary antibodies (TGFβ1, VEGF, COL1A1, COL1A3, NF-KB, and TNFα) were diluted in the blocking buffer solution.
2. The membrane was removed from the blocking buffer and incubated with the primary antibodies overnight at 4° C. with gentle agitation.<

### Secondary Antibody Application:

1. The primary antibody solution was discarded, and the membrane was washed with washing buffer (10 mM Tris pH 7.5, 100 mM NaCl, 0.1% Tween 20) and agitated for 30 minutes. The washing buffer was changed every 10 minutes, and this process was repeated 3 times.
2. Secondary antibodies were diluted in washing buffer as recommended by the manufacturer.
3. The washing buffer was discarded, and the secondary antibody solution was added. The membrane was incubated with the secondary antibody solution at 37° C. for 30 minutes or at room temperature for one hour.

### Chemiluminescence Imaging:

1. After incubation, the petri dish containing the membrane was emptied of the secondary antibody solution, and washing buffer was added to the petri dish. The membrane was washed with agitation for 30 minutes, changing the washing buffer every 10 minutes, and this process was repeated 3 times.
2. Subsequently, the washing buffer was removed from the petri dish, and the membrane was placed in a clean container containing a chemiluminescence working solution (0.125 mL/cm²). The container was agitated for 1-5 minutes to ensure the solution covered the membrane surface.
3. Finally, the resulting protein bands were visualized using a chemiluminescence imaging device.

The expressions of COL1A1, COL3A1, and TGFβ1 proteins were found to increase dose-dependently with H.E and L.E small RNA applications, and the highest expressions were observed with the combined application of H.E and L.E small RNA (FIGS. 12.A, B, and C). The expressions of NF-KB and TNFα proteins decreased dose-dependently with H.E and L.E small RNA applications, and the combined application of H.E and L.E small RNA resulted in the most significant reduction in protein expressions (FIGS. 13.A, B, and C).

### a) Determination of Melanin Content:

In vitro melanin content experiments of H. cordata cell suspension extract and small RNAs were conducted on B16-F10 melanoma cells using the following procedure:
Day 1: B16-F10 cells were suspended enzymatically (trypsin/EDTA) and seeded into 6-well plates at a density of 1×10⁵ cells per well in a culture medium. Cells were incubated at 37° C. with 5% CO₂ for 24 hours.
Day 2: After the incubation period, the culture medium was aspirated, and α-MSH (100 nM) was applied to all wells except the control wells to stimulate melanin synthesis. Cells were incubated for an additional 24 hours.
Day 3: After incubation, the culture medium was removed, and the wells were washed with PBS. For the control (no α-MSH application) and α-MSH (only α-MSH applied) wells, the culture medium containing 10% FBS was added back. In contrast, the other wells were treated with the designated substance doses and positive control. Plates were incubated for 48 hours.
Day 4: After 48 hours of incubation with the substances, the cells were removed using trypsin/EDTA. Cells were collected from the wells and transferred to centrifuge tubes with 2 mL of DMEM+10% FBS. The tubes were centrifuged at 500 g for 5 minutes, and the supernatant was aspirated using a vacuum. The cell pellets were then treated with 300 μL of a 10% DMSO, 1N NaOH solution, and incubated at 80° C. for 1 hour. After the incubation period, the lysates were transferred to the wells of a 96-well plate, and absorbance readings were measured at 475 nm using a microplate reader.

### b) Determination of Tyrosinase Activity

The in vitro anti-tyrosinase activity of H. cordata cell suspension extract and small RNAs on B16-F10 melanoma cells was conducted using the following procedure:
Day 1: B16-F10 cells were enzymatically removed (trypsin/EDTA) from culture flasks and centrifuged (123 g for 5 minutes). The cells were then resuspended in a culture medium and seeded into 6-well plates at a density of 1×10⁵ cells per well. Cells were incubated at 37° C. with 5% CO₂ for 24 hours.
Day 2: After the incubation period, the culture medium was aspirated, and α-MSH (100 nM) was applied to all wells except the control wells to stimulate melanin synthesis. Cells were incubated for an additional 24 hours.
Day 3: After incubation, the culture medium was removed, and the wells were washed with PBS. For the control (no α-MSH application) and α-MSH (only α-MSH applied) wells, the culture medium containing 10% FBS was added back. In contrast, the other wells were treated with the designated substance doses and positive control. Plates were incubated for 48 hours.
Day 5: Cells were removed from the well surface after incubation using trypsin/EDTA.
Cells were collected from the wells and transferred to centrifuge tubes with 2 mL of DMEM+10% FBS. The tubes were centrifuged at 500 g for 5 minutes, and the supernatant was aspirated using a vacuum.
   Each cell pellet was added to 270 μL of lysis buffer (Buffer 50 mM PBS, pH 7.4, containing 1% Triton X100 and 100 μM PMSF).
   The lysate-containing tubes were centrifuged at 17,500 g for 10 minutes at 4° C.
   Supernatants were collected in microcentrifuge tubes. For each 90 μL supernatant, 10 μL of L-DOPA (2 mg/mL) was added, and the mixture was transferred to a 96-well plate.
   The plate was incubated at 37° C. for 30 minutes, and absorbance readings were taken at 475 nm every 10 minutes for a total of 5 readings.
   The percentages of tyrosinase activity suppression were calculated.
% Tyrosinase Activity: Abs_{control}−Absₛₐₘₚₗₑ×100/Abs_{control}
% Tirozinaz Supression Activity: 100-% Tyrosinase Activity

When evaluating the melanin content and tyrosinase activity experiments conducted with melanoma cells, it was determined that melanin content and tyrosinase activity decreased dose-dependently in cells treated with H.E and L.E small RNAs compared to cells treated with only α-MSH. The lowest melanin content and tyrosinase activity were observed in melanoma cells treated with the combination of H.E and L.E small RNAs (FIG. 14 and FIG. 15).

### c) Determination of Cellular Senescence Effect

The anti-senescence activities of H. cordata cell suspension extract and small RNAs were assessed using the SA-β-Galactosidase staining method. In the experiment, an SA-β-Galactosidase solution prepared in 2 mM MgCl₂, 0.1 X-gal, 5 mM potassium ferrocyanide, 5 mM potassium ferricyanide, 150 mM NaCl, and a 400 mM citric acid/sodium phosphate buffer (pH 6) was used.

Human fibroblast cells were seeded in 25 cm² cell culture flasks at a density of 5×10⁵ cells/flask and incubated for 24 hours. Afterward, the serum-containing medium was removed, and the cells were treated with H. cordata cell suspension extract and/or small RNA doses prepared in a serum-free medium. After 48 hours of incubation, the cells in the flasks underwent the following steps:
Washed twice with PBS (phosphate-buffered saline).
Incubated with 4% paraformaldehyde solution for 5 minutes at room temperature.
Washed with PBS two times.
Treated with 2 mL of SA-β-Galactosidase staining solution and incubated with the stain for 12-16 hours.

Cells produce lysosomal β-galactosidase enzyme as a result of metabolic activity. As cells age, lysosomal mass increases, leading to an increase in lysosomal β-galactosidase levels. The activity of the β-galactosidase enzyme in the cells is determined using the chromogenic substrate 5-bromo-4-chloro-3-indolyl β-D-galactopyranoside (X-gal). SA-β-Gal-positive cells appear blue-green under a light microscope. Compared to the control group, cells treated with hydrogen peroxide (H₂O₂) showed increased senescence. However, when H.E and L.E small RNAs were applied in addition to H₂O₂, the blue-green appearance associated with senescence significantly decreased. Moreover, when H.E and L.E small RNAs were applied together, the blue-green color, an indicator of senescence, decreased most significantly in a dose-dependent manner (FIG. 16).

### d) Determination of Protective Effect Against DNA Damage

The protective effects of H. cordata cell suspension extract and small RNAs against DNA damage were determined using the Comet method. The steps of the Comet method are explained below:

A frosted slide was cleaned and dried before use with ethanol. The slide was dipped into low-melting-point agarose (LMP) and then wiped with a cloth to remove excess agarose. The slides were left to dry at room temperature overnight. The cells were removed from culture flasks with trypsin to achieve a cell density of 1×10⁶ cells/mL. Then, 10 μL of cell suspension and 120 μL of LMP agarose were mixed and added to the agarose-coated slides prepared the night before. The slides were covered with a coverslip. After the LMP agarose solidified, the coverslip was removed. The slides were carefully placed in lysis solution, which was previously kept at 2-8° C. for 1 hour to be chill ed. The slides were left in lysis solution for at least 1 hour and a maximum of 24 hours. Afterward, the slides were placed in an electrophoresis tank and covered with an electrophoresis solution. The electrophoresis solution covered the slides up to 1-2 mm from the top. The slides were placed near the anode in the electrophoresis tank. Electrophoresis was conducted at 25 V and 300 mA at 2-8° C. for 30 minutes. After electrophoresis, the slides were treated with neutralization solution twice for 5 minutes each. Then, 100 μL of PI dye (10 μg/mL) was added directly onto the slide and left for 20 minutes. Excess dye was removed by washing. The slides were viewed and examined under a fluorescent microscope.

Evaluation of the visuals obtained from the Comet experiment revealed that the cells treated with MMC (mitomycin C) along with H.E and L.E small RNAs showed shorter DNA tails than cells treated only with MMC, indicating a reduction in DNA damage. Additionally, when H.E and L.E small RNAs were applied together, the images obtained from the cells showed a significantly reduced DNA tail associated with DNA damage, resembling the control group (FIG. 17).

### e) Determination of Protective Effect Against Reactive Oxygen Species (ROS)

The effect of H. cordata cell suspension extract and small RNAs on intracellular ROS (Reactive Oxygen Species) formation was analyzed using the DCFH-DA (2′,7′-dichlorofluorescein diacetate) method. The principle of the DCFH-DA method is based on the diffusion of DCFH-DA into the cell, where cellular esterases deacetylate it into a non-fluorescent compound. This compound is oxidized to a high-fluorescence DCF (2′,7′-dichlorofluorescein) by ROS. Thus, increasing green fluorescence intensity can show the intracellular ROS level. The steps for determining intracellular ROS are described as follows:

DCFH-DA dye was prepared in DMSO to achieve a final concentration of 10 mM. HaCat cells were seeded into a 6-well plate at a density of 6×10⁵ cells/well and incubated in a 5% CO₂ environment at 37° C. After 24 hours, all wells except the control wells were treated with 600 μM H₂O₂, and the cells were incubated with the substance for 6 hours. After the incubation period, the cells were treated with H.E and/or L.E small RNAs or only with the culture medium, and the cells were further incubated for 24 hours. Following the incubation, all wells were washed with 500 μL of PBS, and then 500 μL of 10 μM DCFH-DA was added to the wells. The plate was then incubated in the dark at 37° C. for 45 minutes. After incubation, the wells were rewashed with PBS. Next, the cells were viewed under a fluorescent microscope equipped with a GFP filter, and images were taken.

The fluorescent microscopy results indicated that the application of H.E and L.E small RNAs reduced intracellular ROS levels in a concentration-dependent manner. When H.E and L.E small RNAs were applied together, intracellular ROS formation was significantly suppressed, resembling the control group (FIG. 18).

### f) Determination of Dark Ring Antagonistic Activity Using the Griess Method:

The effects of H. cordata cell suspension extract and small RNAs on dark ring antagonistic activity were determined using the Griess method. The principle of the Griess test is based on the reaction of nitrite with a primary aromatic amine to form a diazonium salt in an acidic solution. Nitrites react with sulfanilic acid in acetic acid to form a diazonium ion, which then reacts with α-naphthylamine to produce a red azo dye. The steps of the Griess method are described below:

The Griess reagent was prepared in distilled water containing 1% sulfanilamide, 0.1% NEDD (1-naphthyl ethylenediamine), and 5% phosphoric acid. A 10 mM Nitrite solution was prepared in distilled water to establish a standard curve. Standard concentrations (5-160 μM) were prepared by diluting the main stock nitrite solution in PBS. THP-1 human monocyte cells were seeded in a 24-well plate at 5×10⁴ cells/well density. To differentiate the cells into macrophages, 5 ng/mL of PMA was added to the wells, and the cells were incubated for 24 hours. After differentiation, 1 μg/mL of LPS (lipopolysaccharide) was added to the culture medium to induce inflammation, and the cells were further incubated for an additional 24 hours. Then, the supernatants from the wells were collected into tubes using a micropipette. 100 μL of each supernatant and standard concentrations were transferred to the wells of a 96-well plate. Subsequently, 100 μL of the Griess reagent was added to all wells, and the plate was incubated at room temperature for 10 minutes. After the incubation period, the absorbance of the plate was measured at 540 nm using a microplate reader.

Based on the obtained absorbance results, it was determined that, compared to the control group, the application of H.E and L.E small RNAs in addition to LPS stimulation reduced nitrite formation. Moreover, when H.E and L.E small RNAs were applied together, nitrite formation was significantly decreased (FIG. 19).

1. The invention relates to a method for producing a cosmetic active ingredient with anti-aging, wound-healing, anti-spot, anti-dark-circle, and anti-hair loss activities, to be used in skin and hair care products. The following steps characterize the method:
obtaining cell suspensions from H. cordata leaves and/or petioles, roots, or stems,
adding 0.05-0.5 mM MeJA to the culture medium of H. cordata cell suspension on the 12th-18th day of culture,
incubating the culture with MeJA for 60-80 hours,
isolating EVs from the culture medium after incubation,
isolating small RNAs from EVs,
encapsulating EV-derived small RNAs in cationic liposomes,
It is characterized by comprising the steps of using the obtained encapsulated small RNAs and/or cell suspension extract as the raw material.

2. The method for producing the cosmetic active ingredient according to claim 1, characterized by the use of Houttuynia cordata or a combination of Cydonia oblonga, Malva sylvestris, Persea americana, Rosa damascena, Helichrysum arenarium, Glycine max, Prunella vulgaris, Symphytum officinale, Malva sylvestris, Scutellaria baicalensis, Pelargonium graveolens, Filipendula ulmaria, Passiflora edulis, Marribium vulgare, Linum tauricum, Lycium barbarum, Papaver rhoeas, Paeonia peregrina, Leontopodium alpinum, Prunus dulcis, Inula helenium, or Psoralea corylifolia plants in the production of the raw material.
